(19) United States
(12) Patent Application Publication
(10) Pub. No.: US 2008/0067079 A1
(43) Pub. Date: Mar. 20, 2008

(54) Method of Analyzing Sample and Analyzing Apparatus

(76) Inventors: Katsuyoshi Takahashi, Tokyo (JP); Yutaka Unuma, Matsudo-shi (JP); Yuji Maruo, Nagareyama-shi (JP)

Correspondence Address: NIXON & VANDERHYE, PC, 901 NORTH GLEBE ROAD, 11TH FLOOR, ARLINGTON, VA, 22203, US

(21) Appl. No.: 11/852,376

(22) Filed: Sep. 10, 2007

(30) Foreign Application Priority Data
Sep. 13, 2006 (JP) 2006-248557

(51) Int. Cl. G01N 27/26; G01N 27/30; G01N 33/00

(52) U.S. Cl. 205/775; 204/400; 204/408; 73/866

(57) ABSTRACT

A sample analysis method of the present invention is a method of analyzing a sample plural times continuously, the sample containing plural sample components, the method including: performing first analysis with the sample in a first analysis medium; collecting the sample components after the first analysis with an analysis result of the first analysis maintained; and providing the collected sample components to second analysis. A sample analysis apparatus of the present invention is an apparatus for analyzing a sample in plural times continuously, the sample containing plural sample components, the apparatus including: a first analysis section in which a first analysis medium is held; a sample collecting section for collecting sample components analyzed by the first analysis; and a second analysis section for analyzing the sample components collected by the sample collecting section. The sample analysis method apparatus of present invention make it possible to analyze sample plural times efficiently.

This Nonprovisional application claims priority under 35 U.S.C. § 119(a) on Patent Application No. 248557/2006 filed in Japan on Sep. 13, 2006, the entire components of which are hereby incorporated by reference.

### FIELD OF THE INVENTION

The present invention relates to a sample analyzing method and analyzing apparatus for analyzing a biological sample, more specifically, to a method, apparatus, and constituent tool, each of which makes it possible to use a sample continuously in plural times of analysis.

### BACKGROUND OF THE INVENTION

After the end of human genome project, many proteome researches have been carried out. The “proteome” means the whole proteins produced by translation in particular cells and organs. Profiling (analysis) of a protein is one of such proteome researches.

Proteome include various proteins. Thus, the proteome researches require a method of analyzing proteins with high separating ability and high sensitivity. In the proteome research, therefore, analysis of components (various proteins) in a sample is carried out generally by using a separating method and a detecting method in combination, which relay on the characteristics of the proteins.

Two-dimensional electrophoresis is one of methods that continuously analyze one sample plural times, the sample containing plural kinds of protein components. Different proteins have different electric charges and molecular weights. Thus, the separation of proteins by using the 2-dimensional electrophoresis is suitable for the analysis of the proteome, which is a mixture of various proteins. That is, by utilizing the differences of the proteins in electric charges and molecular weights in combination, the proteins can be separated with higher separating ability than in the case where the proteins are separated by using the differences of the proteins either in electric charges or in molecular weights.

The 2-dimensional electrophoresis includes two electrophoresis steps, one of which uses isoelectric focusing electrophoresis for separating proteins according to their electric charges, and the other one of which uses molecular weight fractionation electrophoresis (especially, SDS-PAGE) for separating proteins according to their molecular weights. Moreover, the 2-dimensional electrophoresis is such an excellent method that can be used in the presence or absence of a denaturing agent and can separate several hundred kinds of proteins at once (For example, see the Japanese Patent Application Publication, Tokukaihei, No. 11-30605 (published on Feb. 2, 1999)).

Another technique for analyzing proteins is a technique called capillary electrophoresis, in which proteins are separated in a liquid filled in a capillary. Depending on the liquid filled in the capillary, the capillary electrophoresis is called as capillary isoelectric focusing electrophoresis, zone electrophoresis, capillary isotachophoresis, micelle electrokinetic chromatography, or the like. That is, it is possible to separate proteins according to different characteristics by using different solutions as the separating medium.

The capillary electrophoresis uses a solution as the separating medium for the protein. This gives the capillary electrophoresis a very short separating time compared with a gel electrophoresis, which uses, as the separating medium, a gel whose resistance coefficient is large. Among the capillary electrophoresis techniques, the capillary isoelectric focusing electrophoresis can concentrate (separate) the sample components (e.g., proteins) in very narrow ranges according to the electric charges of the samples components. Therefore, the capillary isoelectric focusing electrophoresis is highly regarded as a method of separating, at high sensitivity, compounds (such as proteins) containing many kinds of molecules of both the polarities.

However, the use of the liquid separating medium gives the capillary electrophoresis such a disadvantage that the concentrated proteins are dispersed when voltage application on the separating medium is stopped. This makes it difficult to collect the sample components without disturbing the band pattern in which the sample components are concentrated in the narrow ranges. Thus, it is difficult to combine the capillary electrophoresis with another analysis technique. As one solution of this difficulty, the Japanese translation of PCT patent application, Tokuhyo, No. 2005-517954 (published on Jun. 16, 2005) discloses an art, in which the medium is dried after the electrophoresis so as to keep the separated proteins at theirs positions as they are separated, and then mass spectrometry of the protein thus held at the positions is performed by ionizing the proteins by laser radiation.

In the method in which the gel electrophoresis is used for separating the proteins, it is easy to keep the separated proteins at theirs positions as they are separated. However, the gel separating medium has a large resistance coefficient, which requires a long time to separate (migrate) the proteins. For example, in the isoelectric focusing gel electrophoresis, which separates the proteins solely based on their electric charges, electrical mobility (electric charge amount) of the proteins is small with respect to theirs sizes. Because of this, the protein separation of the isoelectric focusing gel electrophoresis requires a high voltage application and a long analysis time (for example, it requires a voltage application of 8000V for an analysis time of about 8 hours).

In case where the protein analysis is carried out by using, for example, the 2-dimensional electrophoresis described in the Japanese Patent Application Publication, Tokukaihei, No. 11-30605 as the method of separating proteins, in combination with another analysis method, the analysis would take at least half to one day to finish. That is, the method of separating proteins by using the conventional 2-dimensional electrophoresis is low in throughput and is not preferable as a method for proteome analysis, as which should perform plural times of analysis with plural samples.

On the contrary, the capillary isoelectric focusing electrophoresis has a short separating time (about 5 minutes), because it uses a liquid separating medium, whose resistance coefficient is small. However, when the voltage application on the liquid is stopped, the small resistance coefficient also allows the diffusion of the sample components (individual proteins) from where the sample components are migrated by the electrophoresis. That is, the capillary isoelectric focusing electrophoresis has a difficulty in collecting the sample components without disturbing the pattern in which the sample components are concentrated according to the isoelectric point.

For example, in the method described in the Japanese Translation of PCT international application, Tokuhyo, No. 2005-517954, the electrophoresed sample components are kept in a migration vessel by drying. This limits analysis methods combinable with this method. For example, the capillary isoelectric focusing electrophoresis, which can be combined with mass spectrometry, cannot be used in combination with such analysis techniques as gel electrophoresis (SDS-PAGE or the like), chromatography, western blotting, protein analysis using affinity binding reaction (immunity reaction or the like).

Moreover, this method requires much manual works for performing the analysis and preparation thereof. Consequently, there is a limit in the kinds of the analysis that can be continuously performed in one day. In case where the protein analysis is performed by SDS-PAGE and western blotting in combination, the protein detection takes about 2 days. Moreover, to test more samples, it is necessary to do more works and, consequently, more time. In such a case, unexpected mistakes would happen such as mix-up of the samples.

As described above, there are many useful analysis techniques for analyzing samples, but the combinational use of plural kinds of analysis techniques face such problems as (1) it requires more time as the number of analysis techniques to apply is increased, (2) it is difficult to collect the sample components in order to reuse them in the next analysis, and (3) the increase in the number of analysis techniques to apply complicates the operation of the analysis.

### SUMMARY OF THE INVENTION

The present invention was accomplished in view of the aforementioned problems, and an object of the present invention is to provide a sample analysis method and an analysis apparatus, each of which can analyze one sample plural time efficiently, the sample containing plural components.

In order to attain the object, the sample analysis method according to the present invention is a method of analyzing a sample plural times continuously, the sample containing plural sample components, the method including: performing first analysis with the sample in a first analysis medium; collecting the sample components after the first analysis with an analysis result of the first analysis maintained; and providing the collected sample components to second analysis.

In the step of collecting, the sample collecting section is inserted or contacted with the first analysis medium thereto collect in the sample collecting section the sample component analyzed by the first analysis. Moreover, the sample analysis analyzed by the first analysis can be collected in the sample collecting section with the result of the analysis maintained. Thus, simply by moving the sample collecting section, the sample components analyzed by the first analysis can be provided to the second analysis with the result of analysis maintained.

By using an apparatus which can perform each step automatically, it is possible to analyze one sample plural times continuously with a little need of manual operation. Thus, it is possible to obtain analysis result with high reproducibility even among different users.

With this arrangement, it is possible to analyze one sample plural times continuously and efficiently.

Additional objects, features, and strengths of the present invention will be made clear by the description below. Further, the advantages of the present invention will be evident from the following explanation in reference to the drawings.

### BRIEF DESCRIPTION OF THE DRAWINGS

FIG. 1(a) is a perspective view illustrating a whole configuration of a sample analysis apparatus of one embodiment according to the present invention. FIG. 1(b) is an upper-side view illustrating a main configuration of the sample analysis apparatus of the embodiment according to the present invention.

FIG. 2(a) is a perspective view illustrating a whole configuration of a sample analysis apparatus of one embodiment according to the present invention. FIG. 2(b) is an upper-side view illustrating a main configuration of the sample analysis apparatus of the embodiment according to the present invention.

FIG. 3(a) is a perspective view illustrating a whole configuration of a sample analysis apparatus of one embodiment according to the present invention. FIG. 3(b) is an upper-side view illustrating a main configuration of the sample analysis apparatus of the embodiment according to the present invention.

FIG. 4(a) is a perspective view illustrating a main configuration of a sample analysis apparatus of one embodiment according to the present invention. FIG. 4(b) is an upper-side view illustrating a main configuration of the sample analysis apparatus of the embodiment according to the present invention.

FIG. 5(a) is a perspective view illustrating a main configuration of a sample analysis apparatus of one embodiment according to the present invention. FIG. 5(b) is an side view illustrating a main configuration of the sample analysis apparatus of the embodiment according to the present invention.

FIG. 6(a) is a side view illustrating a main configuration of a sample analysis apparatus of one embodiment according to the present invention. FIG. 6(b) is a side view illustrating a main configuration of the sample analysis apparatus of the embodiment according to the present invention. FIG. 6(c) is a side view illustrating a main configuration of the sample analysis apparatus of the embodiment according to the present invention.

FIG. 7(a) is an upper-side view illustrating a main configuration of a sample analysis apparatus of one embodiment according to the present invention. FIG. 7(b) is an upper-side view illustrating a main configuration of the sample analysis apparatus of the embodiment according to the present invention.

### DESCRIPTION OF THE EMBODIMENTS

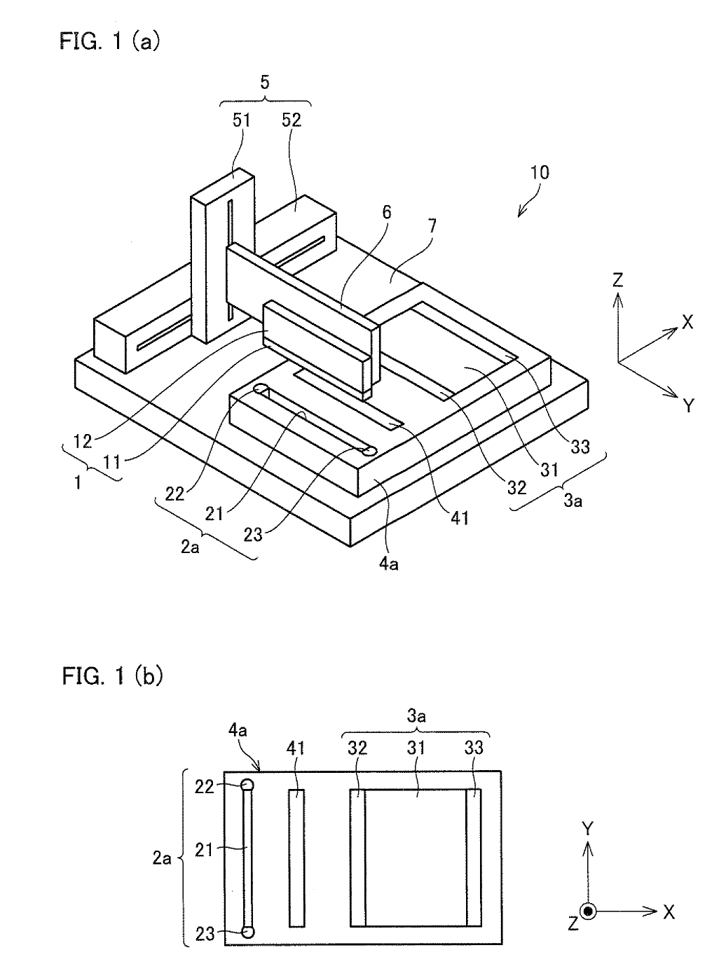

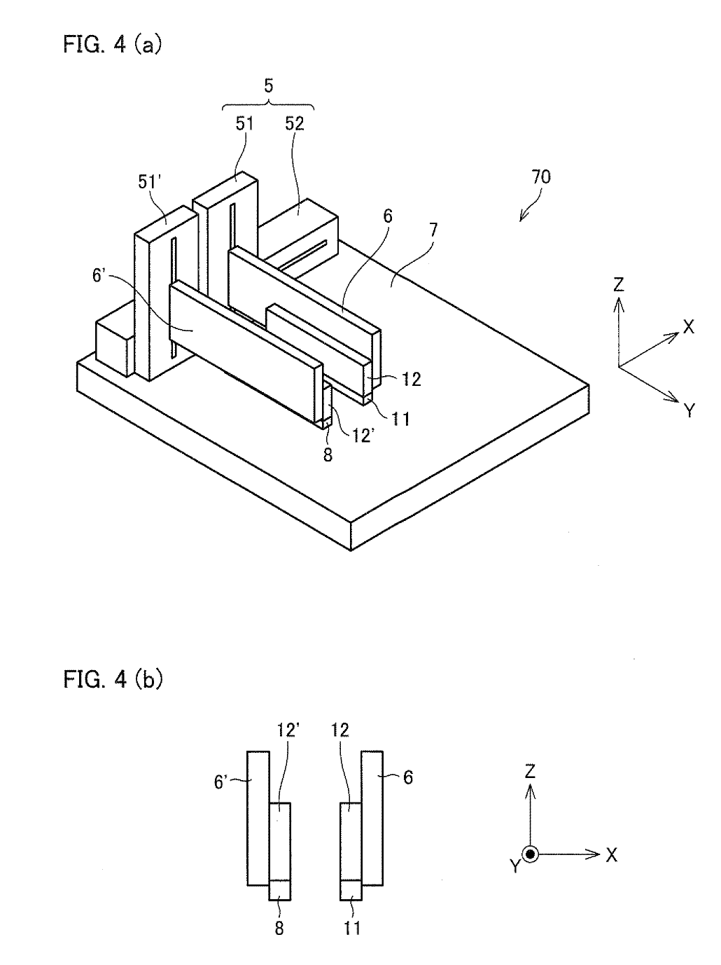

Embodiments of the present invention are described below referring to FIGS. 1 to 7.

[1: Sample Analysis Method]

A sample analysis method according to the present invention is a method of analyzing, by plural times of analysis, a sample containing plural components, the method including the first analysis step of performing first analysis with a sample in a first analysis medium; the collecting step of collecting in a sample collecting section the sample used in the first analysis; and the providing step of providing the collected sample to second analysis.

The term “sample” is used synonymously with “unconditioned sample” and “conditioned sample”. In this Specification, what is meant by the term “sample” is a “biological sample” or an equivalent thereof. The “biological sample” may be any conditioned sample, which is obtained from a biological material (for example, individual, body fluid, cell strain, incubated tissue, or tissue segment) as a supply source. The biological sample may be a body fluid (e.g., blood, saliva, dental plaque, blood serum, blood plasma, urine, synovial fluid, spinal fluid), and a material prepared from a tissue. A preferable biological sample is a sample obtained from a subject. Preferable samples of a subject include an skin lesion portion, sputum, pharyngeal mucus, nasal cavity mucus, pus, and a secreted material. In this Specification, the term “tissue sample” may be a biological sample from a tissue supply source. A living tissue sample and a body fluid can be obtained from a mammal by using any well known method in this field. In this Specification, the term “sample” may be a protein sample, gene DNA sample, or total RNA sample extracted from the biological sample or the tissue sample, besides the biological sample and the tissue sample. Moreover, various factors constituting the “sample” are referred to as “components” or “sample components”. Fractions each including a sample component are collectively referred to as a sample if necessary.

In this Specification, the term “sample analysis” is used to mean at least qualitatively or quantitatively identifying the components (sample components) contained in the sample. The term “sample analysis” may mean analysis that targets a component individually or analysis that targets plural components. The term “sample analysis” encompasses “sample separation”, and may be replaced with a term “analysis of sample components”.

What is meant by the term “collecting of sample” is to capture the sample components in a sample collecting section physically, or to bind or adsorb the sample components with a sample collecting section, thereby to keep the sample components in the sample collecting section.

“To capture the sample components in a sample collecting section physically” may be carried out by using the sample collecting section to absorb a liquid in which the sample components are dissolved or dispersed. In this arrangement, the sample collecting section may be constituted of or include (or be provided with) an absorbing material, which can absorb aqueous liquid or non-aqueous liquid. The absorbing material may be a dry gel, gel, pulp material, filter paper, sponge, cotton, or the like. Among the absorbing materials, the dry gel is preferably used to structure the sample collecting section.

Various conventionally know gels can be used as the raw material from which the sample collecting section is made. Examples of raw materials of the gels encompass acrylamide, N,N′-dimethylacrylamide, N-isopropylacrylamide, agarose, polyvinylalcohol, methylcellulose, poly N-acryloylaminocthoxyethanol, poly N-acryloylaminopropanol, and the like.

Moreover, the principle of the bonding or adsorption of the sample components with the sample collecting section may be use of electric charges, hydrophobic property, hydrophilic property, reactivity with a particular material, or the like property of the sample components, and any combination thereof. The sample collecting section may be constituted of or include (or be provided with) a material selected in accordance with the property of the sample components, or may be given a property that makes it possible to collect the sample components.

For example, in case where the sample components having electric charges, the sample collecting section may be constituted of or include (or be provided with) an electrostatic material such as a material having an electric charge, or may be made electrostatic such as being electrically charged. In this case the sample components and the sample components are bound with the sample collecting section via an electrostatic bonding such as ionic bonding, hydrogen bonding, or the like. Examples of such an electrostatic material encompass a polymer film made or PVDF (Polyvinylidene Difluoride), nitrocellulose, nylon, Teflon (registered trademark), Zitex, polypropylene, polytetrafluoride ethylene, cellulose acetate, latex, or the like. Among all, it is preferable to structure the sample collecting section with a polymer film made of PVDF or nitrocellulose.

Moreover, for example, in case where the hydrophobic sample components are to be collected, the sample collecting section may be constituted of or include (or be provided with) the hydrophobic material, or may be made hydrophobic. In this case, the sample components are bound with the sample collecting section via a chemical bonding such as a hydroscopic bonding. Examples of the hydroscopic material encompass the polymer films made of the materials mentioned above, glass, and substrate materials such as quartz, PMMA (Polymethylmethacrylate), PDMS (polydimethylsiloxane), polyethylene, polystylene, PET (polyethylene terephthalate), COP (cyclic olefin polymer), polycarbonate, vinyl chloride, stainless steel, DLC (Diamond Like Carbon), ceramics, and the like. Among these, the polymer films made of PVDF or nitrocellulose and the substrate materials made of glass, quarts, PMMA, PDMS, or the like are preferable to structure the sample collecting section.

The making the sample collecting section hydrophobic may be carried out by applying a hydrophobic solution (e.g., treating with a silane coupling agent such as hexamethyldisilazane (HMDS), octadecyltrimethoxysilane, or the like), preparing a hydrophobic film by using plasma polymerization (plasma treatment using HMDS), or the other method.

Furthermore, in case where the hydrophilic sample components are collected, the sample collecting section may be constituted of or include (or be provided with) a hydrophilic material or may be made hydrophilic. Examples of the hydrophilic material encompass the polymer film made of cellulose or the like.

The making the sample collecting section hydrophilic or providing the hydrophilic material to the sample collecting section may be carried out by applying a hydrophilic solution, treating with an acid (e.g., sulfuric acid or the like), preparing a hydrophilic film by plasma polymerization (e.g., atmospheric pressure plasma treatment in the presence of oxygen) (e.g., converting methyl group to hydrophilic carboxylic group by using oxygen plasma treatment), making hydrophilic by using covalent bond, or the like method.

Examples of the hydrophilic solution encompass non-ionic surfactant (e.g., glycerin fatty acid ester, sorbitan fatty acid ester, sucrose fatty acid ester, alkyl ethoxylate, Nonylphenol ethoxylate, PEG (polyethyleneglycol), Tween-20, etc.) and phospholipid (e.g., phosphorylcholine, etc.)

To give the hydrophilicity by using covalent bonding may be carried out, for example, by a method including the steps of: washing the sample collecting section with NaOH, HCL, or the like; treating the washed sample collecting section with 3-methacryloxypropyltrimethoxysilane (so as to form double bond in the sample collecting section); treating the double-bonded sample collecting section with dimethylacrylamide, TEMED, and APS (so as to polymerize the sample collecting section and the dimethylacrylamide via the double bond formed in the sample collecting section).

To make the sample collecting section hydrophilic or to provide the hydrophilic material to the sample collecting section may be carried out by a conventionally known method, which is selected in consideration of the material constituting the sample collecting section. Moreover, apart from the method mentioned above, any conventionally known method, which can be applicable to the present invention, can be adopted.

Furthermore, for example, in the case of the sample components reactive (affinitive) with a particular material, the sample collecting section may be constituted of or include (or be provided with) a material affinitive with the sample components to be collected. In this arrangement, depending on the material of the sample collecting section, the bonding between the sample components and the sample collecting section may be ionic bonding, hydrogen bonding, bonding due to antigen-antibody reaction, or base pair formation between two nucleic acids having sequences complementary with each other. Examples of the material affinitive with the sample components to be collected encompass an antibody, a nucleic acid, lectin, or the like. Among all, the antibody is preferable as the material affinitive with the sample components to be collected. The antibody may be a monoclonal antibody, polyclonal antibody, nucleic acid ligand, and artificial polypeptide prepared by molecular imprinting method, or the like. The antigen that is recognized by the antibody may be a protein or a peptide, which has a particular structure, which may be (1) three-dimensional structure of the protein or the peptide, (2) a region modified with a phosphate group, (3) a region modified with a sugar chain, or (4) the like.

Moreover, the sample collecting section may be made of a metal, for example. In this case, the sample components are bound with the sample collecting section via adsorption of a functional group(s) of the sample components to the metal or via adsorption or bonding of the sample components to the metal ionically or electrostatically. The metal used in the sample collecting section may be gold or platinum, or may be a metal oxide such as ZrO₂.

Moreover, for example, a functional group may be provided to the sample collecting section so as to make the sample collecting section hydrophobic, hydrophilic, or reactive to a particular material. Examples of the functional group encompass N-hydroxysuccinimidylester group (NHS ester), epoxy group, carbonyldiimidazol group, isothiocyanate group, sulfonylchloride group, maleimide group, iodoacetamide group, disulfide group, alkyl group, and the like.

For example, the functional group may be provided to the sample collecting section by any of the following six methods:

(1) a method of washing the sample collecting section with a piranha solution (a mixture solution of hydrogen peroxide solution and concentrated sulfuric acid), and treating the sample collecting section with aminopropyltriethoxysilane to give an amino group to the sample collecting section;

(2) the method (1) further including reacting the amino group provided in the sample collecting section with a amino group-reacting site (NHS ester or the like) and a reagent having a desired functional group (e.g., cross-linker reagent of PIERCE (NHS-PEG-Maleimide) or the like);

(3) a method of treating the sample collecting section with a silane coupling agent containing a desired functional group (e.g., γ-grycideoxypropyltrimethoxysilane produced by Dow Corning Corporation, or the like agent);

(4) a method of forming carboxyl group in the sample collecting section by using an atmospheric pressure plasma treatment;

(5) the method (4) further including reacting the carboxyl group provided to the sample collecting section with a carboxyl group-reacting site (amino group or the like) and a reagent having a desired functional group (e.g., 1-Ethyl-3-[3-Dimethylaminopropyl]carbodiimide Hydrochloride produced by Dow Corning Corporation, or the like agent); and

(6) a method of giving the sample collecting section a desired functional group by graft polymerization.

Moreover, to provide the material affinitive to the sample components to be collected may be carried out by any of the following three methods:

(7) a method of binding the functional group given to the sample collecting section with the material affinitive with the sample components to be collected;

(8) a method of bonding, via peptide bonding, the carboxylic group or the amino group given to the sample collecting section with a protein affinitive with the sample components to be collected; and

(9) a method of binding, via hydrophobic bonding, the sample collecting section, which is constituted of, includes, or is provided with a hydrophobic material, with the material affinitive with the sample components to be collected.

The methods (1) to (9) may be selected depending on which material is used in the sample collecting section. Moreover, the present invention is not limited to the methods (1) to (9), and may use any conventionally known adoptable method in order to provide the sample collecting section with a functional group or providing the material affinitive with the sample components to be collected.

What is meant by the term “sample analysis” is to obtain detailed information on the components contained in the sample by separating or detecting the sample components based on the properties (e.g., mass, electric charge, hydrophobic property, hydrophilic property, reactivity with a particular material, or the other property) of the sample components. Moreover, the sample analysis may be carried out in an arbitrarily-selected medium and may be any method conventionally known in this field as a sample analysis method.

The first analysis step of the sample analysis method according to the present invention is preferably carried out in a liquid analysis medium. The capillary isoelectric focusing electrophoresis, zone electrophoresis, isotachophoresis, micelle electrokinetic chromatography, and the like are examples of techniques in which a liquid analysis medium is used. Of all, it is preferable to use capillary isoelectric focusing electrophoresis as the technique of the first analysis.

The capillary isoelectric focusing electrophoresis is an analysis method, which can concentrate or separate the sample components in the liquid analysis medium according to the electric charges of the sample components (e.g., protein).

The liquid analysis medium should be a liquid that can form a pH gradient inside the capillary (migration vessel), and may be a mixture solution of ampholytes whose isoelectric points are gradually different. Because a large resistance coefficient of the analysis medium requires a long time to separate the samples, it is preferable that the ampholytes are material small in molecular weight.

By applying a voltage of the order of 500V/cm on the liquid in which the mixture solution of the ampholytes and the sample are mixed, the sample components are concentrated and separated at positions equal to the isoelectric points in the pH gradient within about 1 to 10 minutes. The sample collecting section may be used as a lid to seal the analysis medium in the capillary, thereby preventing evaporation of the analysis medium. Moreover, because the sample components are concentrated and separated at the positions equal to the isoelectric points in the pH gradient, a separation pattern of the sample components will not change however long the voltage application is carried out. Therefore, the collecting the sample components (or inserting a sample analysis section in the analysis medium) can be carried out at any timing after the sample components are separated in a certain pattern. Moreover, the sample collecting section is constituted of, includes, or is provided with a liquid absorbing material, the sample collecting section may be inserted in the analysis medium while the separation of the sample components is being carried out. In such a case, the sample collecting section is constituted of, includes, or is provided with IPG (immobilized pH gradient), preferably.

Moreover, the sample collecting step can be carried out while the voltage is being applied. Therefore, the sample components can be collected in the separation pattern thereof without disturbing the separation pattern due to the diffusion of the sample components.

In case where the first analysis is carried out in the liquid analysis medium, it is preferable that the analysis method further include the evaporating step of evaporating the analysis medium while the sample collecting step is being performed. By carrying out the sample collecting step while evaporating the analysis medium, the sample collecting section can contact with and collect substantially all the sample components contained in the analysis medium. Moreover, the evaporation of the analysis medium concentrates the sample, thereby making it more efficient to collect the sample.

The first analysis step of the sample analysis method according to the present invention may be carried out by using a solid analysis medium. Molecular weight fractionation electrophoresis such as SDS-PAGE, paper chromatography, in which a filter paper is used as the analysis medium, and the like analysis method are examples of the sample analysis method using the solid analysis medium.

To collect the sample components from the solid analysis method is carried out by contacting the sample collecting section with the solid analysis medium. In the case where the solid analysis medium is used, it is preferable that electrodes are provided at the sample collecting section and the analysis medium, respectively. This arrangement makes it possible to collect electrostatic or statically-electrified sample components efficiently. Moreover, if the sample collecting section is constituted of, includes, or is provided with a metal, the sample collecting section may be used as the electrode.

The sample analysis method of the present invention preferably includes the first sample pretreatment step for performing pretreatment of the sample to be analyzed by the first analysis. The pretreatment of the sample may be, for example, pre-treating the sample with a reagent, which makes it easy to collect the sample components from the first analysis medium by using the sample collecting section. The reagent for the pretreatment of the sample may be any reagent that makes it easy to collect the sample components from the first analysis medium by using the sample collecting section. For example, the reagent for the pretreatment of the sample may be a reagent that gives electrostatic property, hydrophobic property, hydrophilic property, or reactivity with a particular material to the sample to be analyzed by the first analysis. Moreover, the reagent for the pretreatment of the sample may be a reagent that puts the sample components in a condition suitable for the first analysis.

The sample analysis method of the present invention preferably includes the second sample pretreatment step for performing pretreatment of the collected sample components to be analyzed by the second analysis. The pretreatment of the collected sample components may be treating the collected sample components with a reagent, which may be a regent for preparing a suitable condition for the second analysis.

The second analysis of the sample analysis method of the present invention may be any method conventionally known in this field. For example, the second analysis may be separation of sample by using electrophoresis, detection or separation of the sample by using affinity binding reaction, mass spectrometry, chromatography, detection of the sample components by using radioactive isotope.

As described above, the sample analysis method of the present invention is arranged such that the sample collecting section is constituted of, includes, or is provided with a material selected suitably for the samples to be collected and the first analysis medium. With this arrangement, it becomes easy to collect the sample components, which have been analyzed by the first analysis, and the collected sample components can be used in the second analysis. Therefore, the multi-component sample can be analyzed continuously in plural times of analysis.

[2: Sample Analysis Apparatus]

Embodiments of an apparatus for carrying out the sample analysis method according to the present invention are described below referring to the drawings. As to the terminology used in this chapter, refer to the explanation on the terminology in Chapter 1 “Sample Analysis Method” above, when needed.

### 2-1: First Embodiment of Sample Analysis Apparatus

One embodiment of the sample analysis apparatus according to the present invention is described below referring to FIG. 1. A sample analysis apparatus 10 according to the present embodiment is an apparatus for continuously carrying out all the steps of a 2-dimensional electrophoresis from its first-stage analysis and second-stage analysis in the single apparatus. A whole configuration is illustrated in FIG. 1(a).

The sample analysis apparatus 10 according to the present embodiment performs the isoelectric focusing electrophoresis in a liquid filled in a first analysis section 2a, and then analyzes the sample components collected in a sample collecting section 11 by SDS-PAGE in a second analysis section 3a. That is, a first analysis medium is a liquid in the present embodiment.

As illustrated in FIG. 1(a), the sample analysis apparatus according to the present embodiment includes a sample collecting tool 1 for collecting the sample components after the first analysis and provides the sample components to the second analysis. The sample collecting tool 1 includes a sample collecting section 11 and a holding section 12. In the present embodiment, the sample collecting section 11 is a dry gel, which is a liquid absorbing material, and has a function of collecting the sample components physically.

The sample analysis apparatus 10 according to the present embodiment further includes a first analysis section 2a and a second analysis section 3a. The first analysis section 2a includes: a first migration vessel 21, in which the first analysis medium is to be introduced, for performing the isoelectric focusing electrophoresis in the liquid; a reservoir 22 for keeping an anode solution; and a reservoir 23 for keeping a cathode solution. The first migration vessel 21 is thin enough to allow the capillary electrophoresis. The second analysis section 3a includes: a section migration vessel 31 provided with a separation gel so as to perform SDS-PAGE of the sample components, which has been analyzed by the first analysis; a reservoir 32 for keeping an anode solution; and a reservoir 33 for keeping a cathode solution. Even though it is not illustrated in FIG. 1(a), a lid may be provided on the second migration vessel 31.

As illustrated in FIG. 1(a), the first analysis section 2a and the second analysis section 3a are provided on a 2-dimensional electrophoresis substrate 4a, which is provided on a plate 7. Moreover, on the plate 7, a driving means 5 is provided for driving the sample collecting tool 1. The driving means 5 includes a vertical direction driving stage 51 and a horizontal driving stage 52. The sample collecting tool 1 is attached to a supporter 6 connected to the driving means 5, whereby the sample collecting tool 1 can be moved in the X and Z directions in FIG. 1(a).

With the sample analysis apparatus 10 of the present embodiment having this arrangement, it is possible to continuously perform all the steps of the 2-dimensional electrophoresis from the first-stage analysis to the second stage analysis by only one apparatus as described below.

Firstly, the sample to be analyzed and the analysis medium are introduced in the first migration vessel 21. The first migration vessel 21 is a vessel for separating the sample components by liquid isoelectric focusing electrophoresis. At each end of the first migration vessel 21, the reservoirs 22 and 23 are respectively formed. The reservoirs 22 and 23 are respectively filled with the anode solution (for example, phosphate solution) and cathode solution (for example, sodium hydrate solution). Moreover, the anode solution and cathode solution are held in the different reservoirs. In order to perform the electrophoresis in the first migration vessel 21, electrodes are respectively inserted in the reservoirs 22 and 23 respectively so as to contact the solution held in the reservoirs, respectively. The electrodes may be fixedly provided to the reservoirs or may be detachable from the reservoirs. Moreover, the electrodes may be made of any conventionally known materials, provided that the electrodes can apply a voltage on the analysis medium in the first migration vessel 21.

By applying a voltage across the electrodes inserted in the reservoirs 22 and 23, the sample components move through the first migration vessel 21, and separated therein according to theirs electric charges (first analysis).

After the first analysis, the supporter 6 is moved in the X direction of FIG. 1(a) by the horizontal direction driving stage 52 of the driving means 5 thereby reaching right above the first analysis section 2a. Then, the supporter 6 is lowered in the Z direction of the FIG. 1(a) by the vertical direction driving stage 51, thereby contacting the sample collecting section 11 of the sample collecting tool 1 with the first analysis medium in the first migration vessel 21. The sample collecting section 11 in contact with the first analysis medium in the first migration vessel 21 collects (absorbs) the sample components together with the first analysis medium, the sample components having been analyzed in the first analysis medium in the first migration vessel 21. Then, the supporter 6 is moved in the X and/or Z direction of FIG. 1(a) by the vertical direction driving stage 51 and/or the horizontal direction driving stage 52, so as to contact the sample collecting section 11 with the separation gel provided in the second migration vessel 31.

The second migration vessel 31 is a vessel for separating the sample components by SDS-PAGE. On each end of the second migration vessel 31, the reservoirs 32 and 33 are provided respectively. In the reservoirs 32 and 33, electrolytes for the electrophoresis (e.g., solutions containing Tris, Glysine, and SDS) are held. In order to carry out the electrophoresis in the second migration vessel 31, electrodes are inserted in the reservoirs 32 and 33, respectively so as to contact with the solutions in the respective reservoirs. The electrodes may be fixedly provided to the reservoirs or may be detachable from the reservoirs. The electrodes may be made of any material conventionally known, provided that the electrodes can apply a voltage on the separation gel of the second migration vessel 31.

By applying the voltage across the electrodes inserted in the reservoirs 32 and 33, the sample components contained in the sample collecting section 11 contacted with the separation gel move through the separation gel according to the electric charges of the sample components with the result of the first analysis maintained. Thereby, the sample components are separated (second analysis).

The second analysis can be more efficiently performed by providing a sample treating vessel 41 between the first analysis and the second analysis, the sample treating vessel 41 performing electrification of the sample components collected in the sample collecting section 11, and reducing the sample components with a buffer solution (a solution containing SDS and DTT (Dithiothreitol)) held therein.

In this embodiment, the collecting of the sample is carried out after the first analysis is finished. However, it is more preferable that the collection of the sample be carried out by contacting the sample collecting section 11 with the sample medium before the first analysis is completely finished (that is, while the voltage of the first analysis is still being applied). In this arrangement, the sample collecting section can collect that part of the sample components, which exists at the analysis medium in contact with the sample collecting section 11. Further, the sample components can be separated more accurately in the sample collecting section 11 (IPG gel) after high-speed separation is performed in the first analysis. Thus, it is possible to perform isoelectric focusing electrophoresis highly accurately in a short time.

In the following, variations of the members constituting the sample analysis apparatus 10 according to the present embodiment are described.

(Sample Collecting Tool 1)

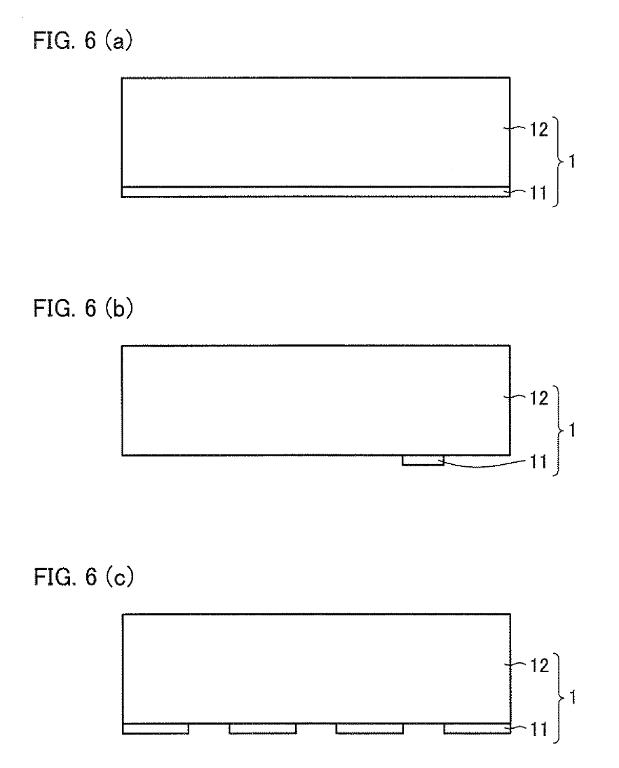

A variation of the sample collecting tool 1 is illustrated in FIG. 6. As illustrated in FIG. 6(a), the sample collecting section 11 is provided preferably over a whole bottom surface of the holding section 12. If necessary, the sample collecting section 11 may have a different size. If a particular fraction of the sample is collected after the first analysis, the sample collecting section 11 may be held at part of the holding section 12, as illustrating in FIG. 6(b). Moreover, as illustrated in FIG. 6(c), plural sample collecting sections 11 may be held on the holding section 12.

In the present embodiment, the sample components are proteins and the first analysis medium is a liquid. Thus, the dry gel, which is an absorbing material, is used as a sample collecting section 11. The material of the sample collecting section 11 may be changed according to the properties of the sample components and the analysis medium as appropriate.

More specifically, another example for analyzing proteins as the sample components to be analyzed, the sample collecting section 11 may be constituted of, include, or be provided with an electrostatic and/or hydrophobic material, because the proteins are electrostatic and/or hydrophobic. Moreover, for example, in the case where the sample components to be analyzed are nucleic acids, the sample collecting section 11 may be constituted of, include, or be provided with an electrostatic and/or hydrophobic material, because the nucleic acids are electrostatic and/or hydrophobic. Moreover, for example, in case where the sample components to be analyzed are lipids, the sample collecting section 11 may be constituted of, include, or be provided with a hydrophobic material, because the lipids are hydrophobic. Moreover, for example, in the case where the sample components to be analyzed are sugars, the sample collecting section 11 may be constituted of, include, or be provided with a hydrophilic material, because the sugars are hydrophilic. In the above 4 examples, the sample collecting section 11 may be provided with a material having such a property appropriate for collecting the sample components or may be converted to have such a property for collecting the sample components.

In case where a functional group bondable with the sample components firmly is attached to the sample collecting section 11, the sample components after the first analysis are bound with the sample collecting section firmly with the analysis result of the first analysis maintained. Therefore, the sample collecting section 11 bound with the proteins firmly can be used as a protein chip, which the sample collecting section 11 bound with the nucleic acid firmly can be used as a DNA chip.

Moreover, the sample collecting section 11 and first analysis medium may be connected with a voltage applying means. This makes it possible for the sample collecting section 11 to collect the sample components from a non-liquid analysis medium after the analysis. In case where the sample collecting section 11 is made of, includes, or provided with a metal, the sample collecting section 11 acts as an electrode.

Moreover, the holding section 12 may be, for example, made of PMMA, polyethylene, PET, or glass. In case where the first analysis is an analysis that requires voltage application, the holding section 12 is preferably made of an electrically insulating material.

The sample collecting tool 1 may have only the sample collecting section 11. However, it is preferable to arrange such that the sample collecting tool 1 is formed by connecting the sample collecting section 11 with the holding section 12, because (1) it is easy to handle a sample collecting section 11 made of a soft material, and (2) it is possible to collect the sample components safely from the first analysis section 2a on which the voltage is applied.

(2-Dimensional Electrophoresis Substrate 4a)

In the 2-dimensional electrophoresis substrate 4a illustrate FIG. 1(b) the first analysis section 2a and the second analysis section 3a are formed on one substrate. However, the first analysis section 2a and the second analysis section 3a may be formed on independent substrates respectively. For example, a substrate on which the first analysis section 2a is formed, and a substrate on which the second analysis section 3a is formed, and a substrate on which the sample treating vessel 41 is formed may be provided instead of the 2-dimensional electrophoresis substrate 4a. Moreover, the sample treating vessel 41 may be formed on the substrate on which the first analysis section 2a or the second substrate section 3a is formed.

The 2-dimensional electrophoresis substrate 4a may be further provided with a sample treating vessel (not illustrated) thereon for pre-treating the sample before providing the sample to the first analysis. Moreover, the 2-dimensional electrophoresis substrate 4a may be further provided with a temperature treating means (not illustrated) thereon for adjusting a temperature of the analysis medium to be introduced into the first analysis section 2a. Moreover, 2-dimensional electrophoresis substrate 4a may be further provided with an electrode (not illustrated) thereon, which is connected with the first analysis medium to be introduced into the first analysis section 2a.

(First Analysis Section 2a)

As described above, the first analysis section 2a of the sample analysis apparatus according to the present embodiment includes the first migration vessel 21, into which the first analysis medium is to be introduced, for performing isoelectric focusing electrophoresis in the liquid; the reservoir 22 for holding the anode solution; and the reservoir 23 for holding the cathode solution.

The analysis medium to be introduced into the first migration vessel 21 should be a liquid that can form a pH gradient when a voltage is applied on the analysis medium. For example, the analysis medium may be a mixture solution of ampholytes whose isoelectric points are different slightly gradually.

Moreover, it is preferable that the first migration vessel 21 has an inner surface treated with acrylamide, hydroxypropylcellulose, methylcellulose, Teflon, polyvinylalcohol, or the like. With this, it is possible to reduce electro osmotic flow, which would disturb a migration pattern when the sample components are migrating.

The first migration vessel 21 is preferably in a range of 1 to 5000 μm in a depth and width (in x-axis direction of FIG. 1). This makes it possible to perform the analysis with a minute amount of the analysis medium, and consequently allows analysis of a sample in a minute amount with high separating ability, and instant absorption or evaporation of the analysis.

As described in the Chapter “1: Sample Analysis Method”, it is preferable to collect the sample while evaporating the analysis medium, in the case of the first analysis that uses liquid analysis medium. As described above, by inserting the sample collecting section 11 in the analysis medium while the voltage application is stilled applied in the first analysis, the sample collecting section 11 can collect that part of the sample components which exists at the position the sample collecting section 11 and the analysis medium are in contact with each other. Further, if the sample is collected with the sample collecting section 11 gradually lowered while the analysis medium is being evaporated, the sample collecting section contacts substantially all the sample components contained in the analysis medium, thereby collecting substantially all the sample components. Moreover, the sample is concentrated by the evaporation of the analysis medium, thereby making it possible to collect the sample more efficiently.

Moreover, the first migration vessel 21 is preferably filled with the analysis medium up to a level in a range of 1 to 1000 μm from its bottom. With this arrangement, it is possible to perform the analysis with a minute amount of the analysis medium. Thus, it is possible to analyze the sample of a minute amount with high separating ability, to perform instant absorption or evaporation of the analysis medium.

The present invention may be arranged such that the first migration vessel 21 is attached with a hydrophilic or hydrophobic material. The hydrophilic or hydrophobic material may be attached to the first analysis section 2a by, for example, application of a hydrophilic solution (non-ionic surfactant, phospholipids, or the like) or a hydrophobic solution to the first migration vessel 21, plasma polymerization to form a hydrophilic film or hydrophobic film on the surface of the first migration vessel 21, or the like method. With this arrangement, it is possible to reduce adsorption or bonding of the sample components to the first migration vessel 21 (1) in case where hydrophobic sample components are to analyzed in the first migration vessel 21 on which the hydrophilic material is attached, or (2) in case where hydrophilic sample components are to analyzed in the first migration vessel 21 on which the hydrophobic material is attached. Thus, this arrangement makes it possible to collect the sample components more efficiently.

The first analysis of the sample analysis apparatus 10 according to the present invention is not limited to the analysis in which proteins are the sample components to be analyzed, or to the use of the liquid analysis medium, even though the above explanation discusses the first analysis in which proteins are sample components to be analyzed and the analysis medium is a liquid. The sample analysis apparatus 10 according to the present invention may be applied to analysis of biological samples such as nucleic acid, sugars, lipids, and the like besides proteins. Moreover, the first analysis may be carried out by various analysis techniques conventionally known. Further, the arrangement of the first analysis section 2a may be changed appropriately according to the analysis technique adopted for the first analysis.

<Second Analysis Section 3a>

As described above, the second analysis section 3a of the sample analysis apparatus 10 according to the present embodiment includes the second migration vessel 31 provided with the separation gel therein for performing SDS-PAGE; the reservoir 32 for holding the anode solution; and the reservoir 33 for holding the cathode solution. The positions of the reservoirs 32 and 33 may be exchanged with each other.

The second migration vessel 31 is provided with the separation gel therein, which acts as the analysis medium. The separation gel may be a conventionally known separation gel such as polyacrylamide gel or the like. The separation gel may be prepared in the second migration vessel 31, or a prepared separation gel is provided in the second migration vessel 31. In the case where the separation gel is prepared in the second migration vessel 31, it is necessary to air-tight the second migration vessel 31. Thus, it is preferable to have the lid on the second migration vessel 31. It is preferable to have a spacer between the lid and the second migration vessel 31 to make the air-tightness.

The reservoir 32 holds the cathode solution and the reservoir 33 holds the anode solution. Electrodes are respectively inserted in the reservoirs 32 and 33. The solution held in the reservoirs 32 and 33 may be, but not limited to, a solution containing SDS, Tris and Glysine.

(Sample Treating Vessel 41)

The sample treating vessel 41 of the present embodiment is provided for providing the sample components to the second analysis (SDS-PAGE) after the sample components are collected from the first analysis (isoelectric focusing electrophoresis) by the sample collecting section 11. More specifically, the sample treating vessel 41 causes the sample to be in equilibrium of the sample (treating the sample with SDS and reducing the sample with a reducing agent). For example, a solution containing Tris-HCl, Glysine, SDS, and DTT may be used for causing the equilibrium of the sample. Note that the present invention is not limited to this solution for causing the equilibrium.

The sample treating vessel 41 may be arranged in any way, provided that the sample treating vessel can give the sample components a condition suitable for the second analysis.

Moreover, the 2-dimensional electrophoresis substrate 4a may be further provided with a sample treating vessel for pre-treating the sample before the first analysis, which sample treating vessel 21 is different from the sample treating vessel 41 for pre-treating the sample before the second analysis. The sample treating vessel for the pretreatment for the first analysis gives the sample components a condition suitable for the first analysis. For example, the sample treating vessel may be a treating vessel for treating the sample thereby to make it possible to collect the sample components from the first analysis medium easily (e.g., making the sample components electrostatic, hydrophilic, hydrophobic, or the like).

(Driving Means 5)

As illustrated in FIG. 1, the sample analysis apparatus 10 includes the driving means 5, which includes the vertical direction driving stage 51 for moving the sample collecting tool 1 in the direction parallel to the Z axis, and the horizontal direction driving stage 52 for moving the sample collecting tool in the direction parallel to the X axis. The vertical direction moving stage 51 is connected to the supporter 6, which holds the sample collecting tool 1 in such a manner that the sample collecting section 11 is in contact with the first analysis section 2a.

The driving means 5 of the sample collecting apparatus 10 makes it possible to move the sample collecting apparatus 1 to a desired position. Furthermore, this arrangement is safer, because it is not necessary to move the apparatus and the member directly. The driving means 5 may be stepping mortar stage, servo stage, or the like, for example.

The supporter 6 may be made of PMMA and Metal, for example. Moreover, the sample collecting tool 1 may be connected to the supporter 6 by holding firmly with vacuum suction, a clamp, an electrostatic force, a magnetic force, adhesion, or the like. In case where the supporter 6 and the sample collecting tool 1 are connected with each other by vacuum suction, a clamp, an electrostatic force, or a magnetic force, the sample collecting tool 1 is detachable from the supporter 6.

<Other Preferable Arrangement>

Examples of the temperature adjusting means for adjusting the temperature of the analysis medium to be introduced in the first analysis section 2a encompass peltier device, heater, temperature measuring device, and the like device. The temperature adjusting means may include plural devices in combination. The temperature adjusting means may be positioned at any location which allows the temperature adjusting means to adjust the temperature of the analysis medium. Moreover, the temperature adjusting means may be provided under or in a vicinity of a region under the first analysis section 2a provided on the plate 7, instead of being provided on the 2-dimensional electrophoresis substrate 4a.

With the arrangement in which the temperature adjusting means is provided on the 2-dimensional electrophoresis substrate 4a, the liquid analysis medium can be evaporated. This makes it possible to concentrate the sample in the liquid medium, thereby making it possible to collect the sample components more efficiently.

It is preferable that the sample analysis apparatus 10 is connected to a power source. This makes it possible to supply power to the first analysis section 2a, the second analysis section 3a, the driving means 5, the temperature adjusting means, and the voltage application means, thereby making it possible to carry out the sample analysis automatically. Moreover, the sample analysis apparatus 10 may be connected to a computer, in which a control program is installed for controlling the analysis procedure of the sample analysis apparatus 10. With this arrangement, the sample analysis apparatus 10 according to the present embodiment can perform the following actions automatically:

separating the sample components (proteins) by the isoelectric focusing electrophoresis performed in the first analysis section 2a;

collecting (absorbing) the separated sample components by the sample collecting section 11;

putting the collecting sample components in equilibrium in the sample treating vessel 41;

transporting the sample in equilibrium to an end of the second migration vessel 41; and

separating the transported sample by SDS-PAGE performed by the second analysis section 3a.

Because all the procedures of the analysis in the sample analysis apparatus 10 can be performed under automatic control, the sample analysis can be carried out with higher safety and reproducibility.

To analyze the sample components after the second analysis, the proteins having been analyzed and held in the sample collecting section 11 may be stained by CBB staining or sliver staining after the above actions.

### 2-2: Second Embodiment of Sample Analysis Apparatus

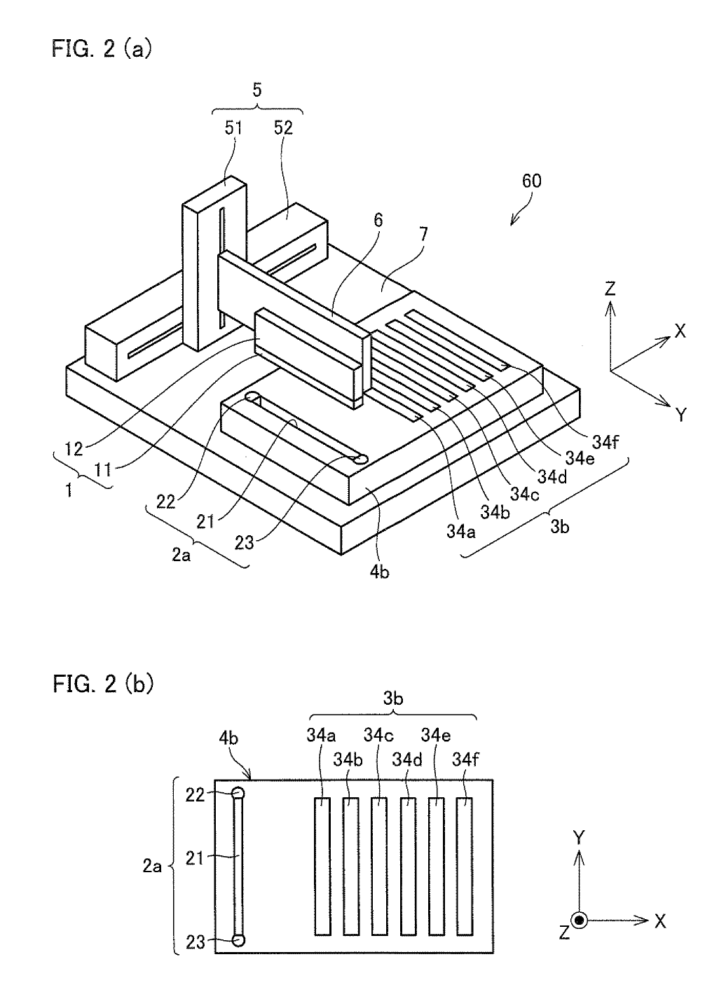

One embodiment of the sample analysis apparatus according to the present invention is described below, referring to FIG. 2. A sample analysis apparatus 60 according to the present invention is an apparatus for performing all steps from a first analysis (isoelectric focusing electrophoresis in a liquid) to a second analysis (western blotting) by one apparatus continuously. A whole configuration is illustrated in FIG. 2(a). In the present embodiment, members identical with those in the first embodiment are labeled in the same manner.

The sample analysis apparatus 60 according to the present embodiment performs the isoelectric focusing electrophoresis of a sample in a liquid introduced in a first analysis section 2a, collects the sample therefrom by a sample collecting section 11, and then analyzes the collected sample by western blotting performed by the second analysis section 3b. That is, the first analysis uses a liquid analysis medium in the present embodiment.

As illustrated in FIG. 2, the sample analysis apparatus 60 includes a sample collecting tool 1 for collecting sample components after the first analysis and proving the collected sample components to the second analysis. The sample collecting tool 1 includes a sample collecting section 11 and a holding section 12. In the present embodiment, the sample collecting section 11 is a PVDF film, which absorbs (collects) the sample components based on the electrostatic property and hydrophobic property thereof, in order to carry out the western blotting in the second analysis.

The sample analysis apparatus 60 according to the present embodiment further includes a first analysis section 2a and a second analysis section 3b. The first analysis section 2a includes a first migration vessel 21, in which a first analysis medium is to be introduced, in order to perform the isoelectric focusing electrophoresis in the liquid; a reservoir 22 for holding anode solution; and a reservoir 23 for holding cathode solution. The locations of the reservoirs 22 and 23 may be exchanged. The second analysis section 3b includes a sample treating vessel 34a for blocking the sample components collected by the sample collecting section 11 in order to detect the collected sample components by the western blotting; a sample treating vessel 34b for washing the blocked sample components; a sample treating vessel 34c for reacting the sample components with primary antibody; a sample treating vessel 34d for washing the sample components reacted with the primary antibody; a sample treating vessel 34e for reacting, with a secondary antibody, the sample components reacted with the primary antibody; and a sample treating vessel 34f for washing the sample components reacted with the secondary antibody.

As illustrated in FIG. 2, the first analysis section 2a and the second analysis section 3b are formed on the substrate 4b disposed on a plate 7. The plate 7 on which substrate 4b is disposed is provided with driving means 5 for moving the sample collecting tool 1. The driving means 5 includes a vertical direction driving stage 51 and a horizontal driving stage 52. The sample collecting tool 1 is attached to a supporter 6 connected to the driving means 5, thereby the sample collecting tool 1 can move in the X and Z directions of FIG. 2(a).

With the sample analysis apparatus 60 according to the present embodiment having this arrangement, it is possible to carry out all the steps from the first analysis to the second analysis by one apparatus continuously as described below.

The first analysis is similar to that in the first embodiment. After the first embodiment, the supporter 6 is moved to a position right above the first analysis section 2a in the X direction of FIG. 2(a) by the horizontal direction driving stage 52 of the driving means 5, and then the supporter 6 is lowered in the Z direction of FIG. 2(a) by the vertical direction driving stage 51 thereby to contact the sample collecting section 11 of the sample collecting tool 1 with the first analysis medium of the first migration vessel 21. The sample collecting section 11 contacted with the first analysis medium of the first migration vessel 21 absorbs (collects) the sample components which has been analyzed in the first analysis medium of the first migration vessel 21. The absorption of the sample components is carried out based on the electrostatic property and hydrophobic property of the sample components. The supporter 6 is moved in the X and/or Z direction of FIG. 2(a) by the vertical direction driving stage 51 and the horizontal direction driving stage 52 thereby to transport the sample collecting section 11 to the second analysis section 3b (34a to 34f). By the driving means 5, the sample collecting section 11 is moved through the vessels 34a to 34f constituting the second analysis section 3b. By this, the western blotting is carried out (second analysis). In the vessels 34a to 34f constituting the second analysis section 3b, solutions (buffer solutions or the like) for carrying out the steps of the western blotting are held. The solution will be described later.

With the sample analysis apparatus according to the present embodiment having this arrangement, it is possible to carry out the steps from the first analysis (isoelectric focusing electrophoresis in the liquid) and the second analysis (western blotting) by one apparatus continuously.

Moreover, even though the present embodiment is arranged such that the sample is collected after the end of the first analysis, it is more preferable that the sample collecting section 1 contact with the analysis medium and collect the sample components before the first analysis is completed finished (that is, while the voltage of the first analysis is applied). In this case, the sample collecting section 11 collects that portion of the sample components that exists at a position in which the sample collecting section 11 and the analysis medium contact with each other. Moreover, as in the first embodiment, it is preferable that the sample is collected while the analysis medium is being evaporated. By collecting the sample by gradually lowering the sample collecting section 11 while evaporating the analysis medium, the sample collecting section 11 contacts with substantially all the sample components contained in the analysis medium. Thereby, it is possible to collect substantially all the sample components. Moreover, the evaporation of the sample analysis concentrates the sample, thereby making it possible to collect the sample more efficiently.

The constituent members of the sample analysis apparatus 60 according to the present embodiment are not limited to what are described above, and may be provided with temperature adjusting means and voltage applying means (not illustrated) as in the first embodiment. In the following, variations of the constituting members are described.

<Sample Collecting Tool 1>

As illustrated in FIG. 6, the sample collecting section 11 in the present embodiment is held on a whole bottom surface of the holding section 12. The sample collecting section 11 is not limited to the PVDF film and may be a film made of nitrocellulose.

<Substrate 4b>

As illustrated in FIG. 2(b), the first analysis section 2a and the second analysis section 3a are formed on one substrate 4b, but may be formed on independent substrates respectively. For example, instead of the substrate 4b, a substrate on which the first analysis section 2a is formed, and a substrate on which the second analysis section 3b is formed may be used.

<Second Analysis Section 3b>

As described above, the second analysis section 3b of the sample analysis apparatus 60 according to the present embodiment includes a sample treating vessel 34a for blocking sample components collected by the sample collecting section 11, so as to detect the collected sample components by the western blotting; a sample treating vessel 34b for washing the blocked sample components; a sample treating vessel 34c for reacting the sample components with primary antibody; a sample treating vessel 34d for washing the sample components reacted with the primary antibody; a sample treating vessel 34e for reacting, with a secondary antibody, the sample components reacted with the primary antibody; and a sample treating vessel 34f for washing the sample components reacted with the secondary antibody.

The sample treating vessel 34a holds a blocking solution containing Bovine Serum Albumin (BSA), PBST (Phosphate Buffer Saline Tween-20), and the like. The sample treating vessel 34c holds a solution containing the primary antibody, which specifically binds with the sample components to be detected. The sample treating vessel 34e is filled with a solution, which contains the secondary antibody, which specifically binds with the primary antibody and is labeled with a fluorescent dye or the like. The sample treating vessels 34b, d, and f are filled with a washing solution made of PBST or the like.

In the present embodiment, the primary antibody is reacted with the collected sample components collected by the sample collecting section 11. However, the present invention may be arranged such that the primary antibody is transferred and blocked in advance, and then collected by the sample collecting section 11. In this case, the second analysis section 3b should include four sample treating vessels 34. Moreover, the sample collecting section 11 in which the sample components after the first analysis are collected can be used as a protein chip.

<Other Preferable Arrangements>

It is preferable that the sample analysis apparatus 60 is connected to a power source. This makes it possible to supply power to the first analysis section 2a, the driving means 5, the temperature adjusting means, and the voltage application means, thereby making it possible to carry out the analysis of the sample components automatically. Moreover, the sample analysis apparatus 60 may be connected to a computer, in which a control program is installed for controlling the analysis procedure of the sample analysis apparatus 60. With this arrangement, the sample analysis apparatus 60 according to the present embodiment can perform the following actions automatically;

separating the sample components (proteins) by the isoelectric focusing electrophoresis performed in the first analysis section 2a;

collecting (absorbing) the separated sample components by the sample collecting section 11;

blocking the collected sample components in the sample treating vessel 34a;

washing the blocked sample components in the sample treating vessel 34b;

causing an antigen-antibody reaction between the washed sample components and the primary antibody in the sample treating vessel 34c;

washing the sample components in the sample treating vessel 34d after the reacting with the primary antibody;

causing an antigen-antibody reaction between the secondary antibody and the primary antibody bound with the sample components; and

washing the sample components after the reaction with the secondary antibody. Because all the procedures of the analysis in the sample analysis apparatus 60 can be performed under automatic control, the sample analysis can be carried out with higher safety and reproducibility.

To analyze the sample components after the second analysis, the secondary antibody may be radiated with a laser light or the like so as to detect the analyzed protein in the sample collecting section 11 according to excitation light after the above actions.

### 2-3: Third Embodiment of the Sample Analysis Apparatus

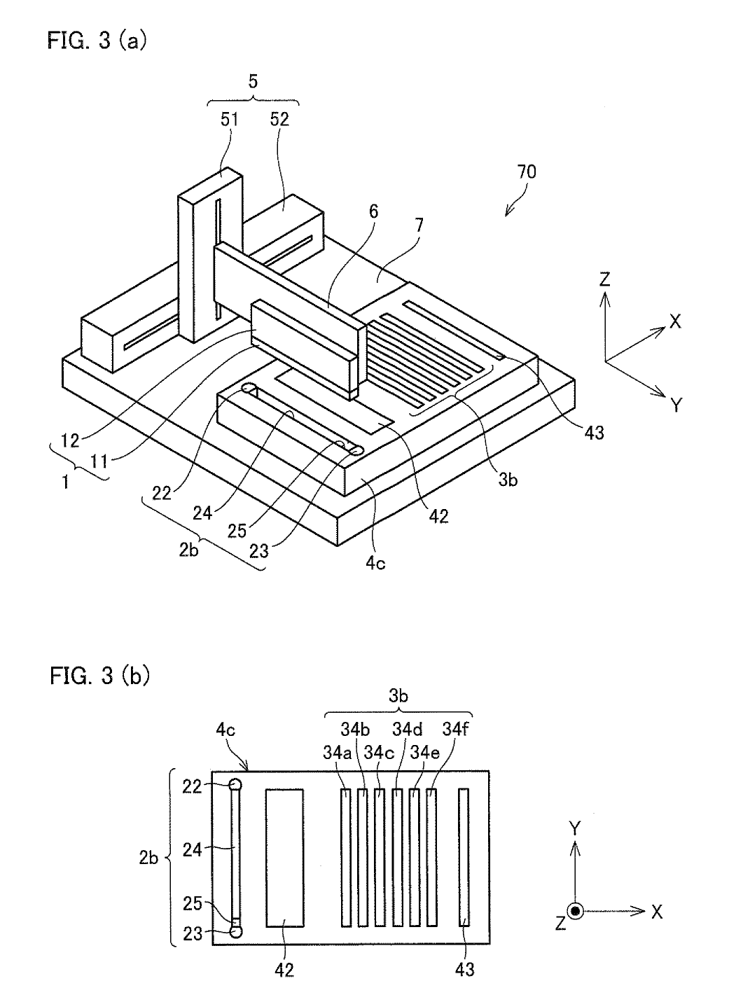

One embodiment of a sample analysis apparatus according to the present invention is described below referring to FIG. 3. A sample analysis apparatus 70 according to the present embodiment is an apparatus for performing all the steps from the first analysis (SDS-PAGE) to the second analysis (western blotting) by one apparatus continuously. A whole configuration of the sample analysis apparatus 70 according to the present embodiment is illustrated in FIG. 3(a). In the present embodiment, members identical with those in the first or second embodiment are labeled in the same manner.

The sample analysis apparatus 70 according to the present embodiment performs the SDS-PAGE of a sample in a first analysis section 2a, collects the sample therefrom by a sample collecting section 11, and then analyzes the collected sample by western blotting. That is, the first analysis uses a solid analysis medium (gel) in the present embodiment.

As illustrated in FIG. 3(a), the sample analysis apparatus 70 according to the present embodiment includes a sample collecting tool 1 for collecting the sample components after the first analysis and providing the collected sample components to the second analysis. The sample collecting tool 1 includes a sample collecting section 11 and a holding section 12. In order to collect, by the sample collecting section 11, the sample components analyzed in the first analysis medium 8, the sample collecting section 11 is a PVDF film, which absorbs (collects) the sample components based on the electrostatic property and hydrophobic property thereof, in order to collect by the sample collecting section 11 the sample components analyzed in the first analysis medium. Moreover, even though it is omitted in FIG. 3(a), the sample analysis apparatus 70 according to the present embodiment includes an analysis medium transporting tool 1′ for transporting the first analysis medium after the first analysis. The analysis medium transporting tool 1′ includes a holding section 12′ the first analysis medium held therein.

As illustrated in FIG. 3(a), the first analysis section 2b includes: a migration vessel 24, provided with the first analysis medium 8, for performing SDS-PAGE for separating the sample components; a reservoir 22 for holding an anode solution; a reservoir 23 for holding a cathode solution; and a sample introducing section 25 for introducing the sample into the first analysis medium 8. The second analysis section 3b includes a sample treating vessel 34a for blocking the sample components collected by the sample collecting section 11 in order to detect the collected sample components by the western blotting; a sample treating vessel 34b for washing the blocked sample components; a sample treating vessel 34c for reacting the sample components with primary antibody; a sample treating vessel 34d for washing the sample components reacted with the primary antibody; a sample treating vessel 34e for reacting, with a secondary antibody, the sample components reacted with the first antibody; and a sample treating vessel 34f for washing the sample components reacted with the secondary antibody.

In order to collect the sample components from the first analysis medium 8 to the sample collecting section 11 based on the electrostatic property of the sample components, the sample analysis apparatus 70 according to the present embodiment further includes a sample transfer vessel 42 filled with a buffer solution for the sample transfer, and a transfer film storage section 43 for storing the sample collecting tool 1 before the analysis.

As illustrated in FIG. 3, the first analysis section 2b, the second analysis section 3b, the sample transfer vessel 42, and the transfer film storage section 43 are provided on a substrate 4c disposed on a plate 7, On the plate 7, driving means including a vertical direction driving stage 51 and a horizontal direction driving stage 52 are disposed.

A supporter 6 connected to the driving means 5 (vertical direction driving stage 51) can move in the X and Z directions of FIG. 3(a). The analysis medium transporting tool 1′ and the sample collecting tool 1, which can be attached to the supporter 6 by vacuum suction, can also move in the X and Z directions of FIG. 3(a), similarly.

With the sample analysis apparatus 70 according to the present embodiment having this arrangement, it is possible to carry out all the steps from the first analysis to the second analysis by one apparatus continuously as described below.

The migration vessel 24 of the first analysis section 2b is a vessel for storing the first analysis medium 8 for separating the sample components by SDS-PAGE. the analysis medium transporting tool 1′ including the first analysis medium 8 and the holding section 12′ is attached to the supporter 6′ by vacuum suction. The supporter 6′ is moved in the X direction of FIG. 3(a) to a position right above the migration vessel 24 by the horizontal direction driving stage 52 of the driving means 5. Then, the supporter 67 is lowered in the Z direction of FIG. 3(a) by the vertical direction driving stage 51. As a result, the first analysis medium 8 of the analysis medium transporting tool 1′ is stored in the migration vessel 24.

On each end of the migration vessel 24, the reservoirs 22 and 23 are provided respectively. In the reservoirs 22 and 23, electrolytes for the electrophoresis (e.g., solutions containing Tris, Glysine, and SDS) are held. In order to carry out SDS-PAGE in the first analysis section 2b, electrodes are inserted in the reservoirs 22 and 23, respectively so as to contact with the solutions in the respective reservoirs. The electrodes may be fixedly provided to the reservoirs or may be detachable from the reservoirs. The electrodes may be made of any material conventionally known, provided that the electrodes can apply a voltage on the analysis medium 8 stored in the migration vessel 24.

The sample introducing section 25 for introducing the sample into the first analysis medium 8 is provided at that end of the migration vessel 24 which is adjacent to the reservoir 23, so as to introduce in the first analysis medium the sample to be analyzed.

By introducing in the sample introducing section 25 the sample to be analyzed and applying a voltage across the electrodes inserted in the reservoirs 22 and 23, the sample components are moved and separated through the first migration vessel 24 according to electric charges of the sample components (first analysis).

After the first analysis, the supporter 6 is moved in the X and/or Z direction of FIG. 3(a) by the vertical direction driving stage 51 and horizontal driving stage 52, thereby to introduce the first analysis medium 8 into the buffer solution for the sample transfer, the buffer solution being held in the sample transfer vessel 42. After the first analysis medium 8 is disposed at a fixing section formed in the sample transfer vessel 42, the vacuum suction is released thereby to separate the analysis medium transporting tool 1′ and the supporter 6.

Next, the driving means 5 moves the supporter 6 above the transfer film storage section 43. Then, the sample collecting tool 1 is adhered to the supporter 6 by vacuum suction. After that, the supporter 6 is moved in the X and/or Z direction of FIG. 3(a) by the vertical direction driving stage 51 and horizontal driving stage 52, thereby to introduce, into the buffer solution for the sample transfer, the sample collecting section 11 of the sample collecting tool 1 adhered to the supporter 6, the buffer solution being held in the sample transfer vessel 42.

As described above, the sample collecting section 11 is a PVDF film. However, it is not necessary that the whole sample collecting section 11 illustrated therein is the PVDF film, provided that the PVDF film is disposed on a surface which will contact with the first analysis medium 8 by moving the supporters 6 and/or 6′ in the X direction of FIG. 3(b).

The sample transfer vessel 42 is provided with electrodes, which moves the sample components from the first analysis medium 8 to the sample collecting section 11 (in the X direction of FIG. 3(b) by the voltage application. That is, the sample transfer vessel 42 is provided with the electrodes facing each other, sandwiching the first analysis medium 8 and the sample collecting section 11 therebetween. The electrode associated with the sample collecting section 11 may be provided on the sample collecting section 11 itself. In this arrangement, the electrode provided to the sample collecting section 11 and the first analysis medium 8 sandwiches the PVDF film therebetween in the X direction of FIG. 3(b).

By applying the voltage across the electrodes provided to the sample transfer vessel 42, the sample components are transferred out of the first analysis medium 8 to the sample collecting section 11 (PVDF film portion) according to the electric charges of the sample components (sample collecting).

After collecting the sample, the supporter 6 is moved in the X and/or Z direction of FIG. 3(a) by the vertical direction driving stage 51 and the horizontal direction driving stage 52, thereby to transport the sample collecting section 11 to the second analysis section 3b (34a to 34f). By the driving means 5, the sample collecting section 11 is moved through the vessels 34a to 34 constituting the second analysis section 3b. By this, the western blotting is carried out (second analysis). In the vessels 34a to 34f constituting the second analysis section 3b, solutions (buffer solutions or the like) for carrying out the steps of the western blotting are held. The solution will be described later.

With the sample analysis apparatus 70 according to the present embodiment having this arrangement, it is possible to carry out the steps from the first analysis (SDS-PAGE) and the second analysis (western blotting) by one apparatus continuously.

The constituent members of the sample analysis apparatus according to the present embodiment are not limited to what are described above, and may be provided with temperature adjusting means and voltage applying means (not illustrated) as in the first embodiment. In the following, variations of the constituting members are described.

(Analysis Medium Transporting Tool 1′)

As described above, the analysis medium transporting tool 1′ includes the first analysis medium 8 and the holding section 12′. The first analysis medium 8 may be adhered to or attached detachably to the holding section 12′. In case where the first analysis medium 8 is attached detachably to the holding section 12′, vacuum suction system may be used. Thus, that surface of the holding section 12′ to which the first analysis medium 8 is attached is flatten and has a sucking hole for the vacuum suction. The vacuum suction of the first analysis medium 8 to the holding section 12′ can be done by connecting with the holding section 12′ a vacuum suction means conventionally known in this field.

A person skilled in the art will easily understand from this Specification that the vacuum suction system is also applicable to attaching the sample collecting section 11 to the holding section 12 in the sample collecting tool 1, attaching the supporter 6 with the sample collecting tool 1, and attaching the supporter 6′ to the analysis medium transporting tool 1′.

<Driving means 5 and Supporter 6>

The present invention may be arranged such that plural vertical direction driving stages 51 and the supporters 6 are provided, even though the present embodiment described above is arranged such that the vertical driving stage 51 and the supporter 6 transport the sample collecting tool 1 and the analysis medium transporting tool 1′ one by one by using the vacuum suction.

In case where the vacuum suction is not used, supporters 6 and 6′ may be used, which sticks with the sample collecting tool 1 and the analysis medium transporting tool 1′ independently. With this arrangement, it is not necessary to have the fixing section to which the first analysis medium 8 is disposed in the sample transfer vessel 42. Moreover, the vertical direction driving stage 51 and the horizontal direction driving stage 52 can easily adhere the sample collecting tool 1 and the analysis medium transporting tool 1′ closely.

<Substrate 4C>

As illustrated in FIG. 3(b), the first analysis section 2b, the second analysis section 3b, the sample transfer vessel 42, and the transfer film storing vessel 43 may be formed on one substrate 4c, but may be provided on separate substrates respectively.

<First Analysis Section 2b>

As illustrated in FIG. 3(b), the first analysis section 2b includes the following elements necessary for separating the sample components: the migration vessel 24 in which the separating gel is to be disposed; the reservoir 22 for holding an anode solution; the reservoir 23 for a cathode solution; and the sample introducing section 25 for introducing the sample into the separating gel. In the present embodiment, the reservoir 23 may function as the sample introducing section 25 as well.

<Sample Transfer Vessel 42>

As described above, the sample transfer vessel 42 is a vessel used for transferring the sample components analyzed by the first analysis, from the analysis medium (separating gel) 8 of the first analysis to the PVDF film of the sample collecting section 11. Therefore, the sample analysis apparatus 70 collects the sample components by using the sample transfer vessel 42 after the first analysis.

As illustrated in FIG. 3(b), the sample transfer vessel 42 is provided between the first analysis section 2b and the second analysis section 3b. The sample transfer vessel 42 should be located at such a position that the supporter 6′ can be transported thereto from the first analysis section 2b by the vertical direction driving stage 51 and the horizontal directing driving stage 52.

Even though it is not illustrated here, a filter paper may be provided (i) between the electrode and the first analysis medium, and/or (ii) between the electrode and the PVDF film, in the buffer solution in the sample transfer vessel 42. The electrodes provided to the sample transferring vessel 42 may be formed in the sample transfer vessel 42 in advance or may be provided when the sample analysis is carried out. The buffer solution for the sample transfer is generally a buffer solution containing Tris, Glysine, and methanol. However, the present invention is not limited to such a buffer solution.

<Other Preferable Arrangements>

It is preferable that the sample analysis apparatus 70 is connected to a power source. This makes it possible to supply power to the first analysis section 2a, the driving means 5, and the sample transfer section 42, thereby making it possible to carry out the analysis of the sample components automatically. Moreover, the sample analysis apparatus 70 may be connected to a computer, in which a control program is installed for controlling the analysis procedure of the sample analysis apparatus 70. With this arrangement, the sample analysis apparatus 70 according to the present embodiment can perform the following actions automatically:

separating the sample components (proteins) according to their molecular weights by SDS-PAGE performed in the first analysis section 2b;

collecting (transferring) the separated sample components by the sample collecting section 11 in the sample transfer vessel 42;

blocking the collected sample components in the sample treating vessel 34a;

washing the blocked sample components in the sample treating vessel 34b;

causing an antigen-antibody reaction between the washed sample components and primary antibody in the sample treating vessel 34c;

washing the sample components in the sample treating vessel 34d after the reaction with the primary antibody;

causing in the sample treating vessel 34e an antigen-antibody reaction between the secondary antibody and the antibody bound with the sample components; and

washing the sample components after the reaction with the secondary antibody.

Because all the procedures of the analysis in the sample analysis apparatus 70 can be performed under automatic control, the sample analysis can be carried out with higher safety and reproducibility.

By appropriately changing the first analysis medium, a detection agent, and the sample collecting section 11, this arrangement also makes it possible to automatically carry out southern blotting or northern blotting of DNA or RNA as the sample components to be analyzed.

### 2-4: Fourth Embodiment of Sample Analysis Apparatus

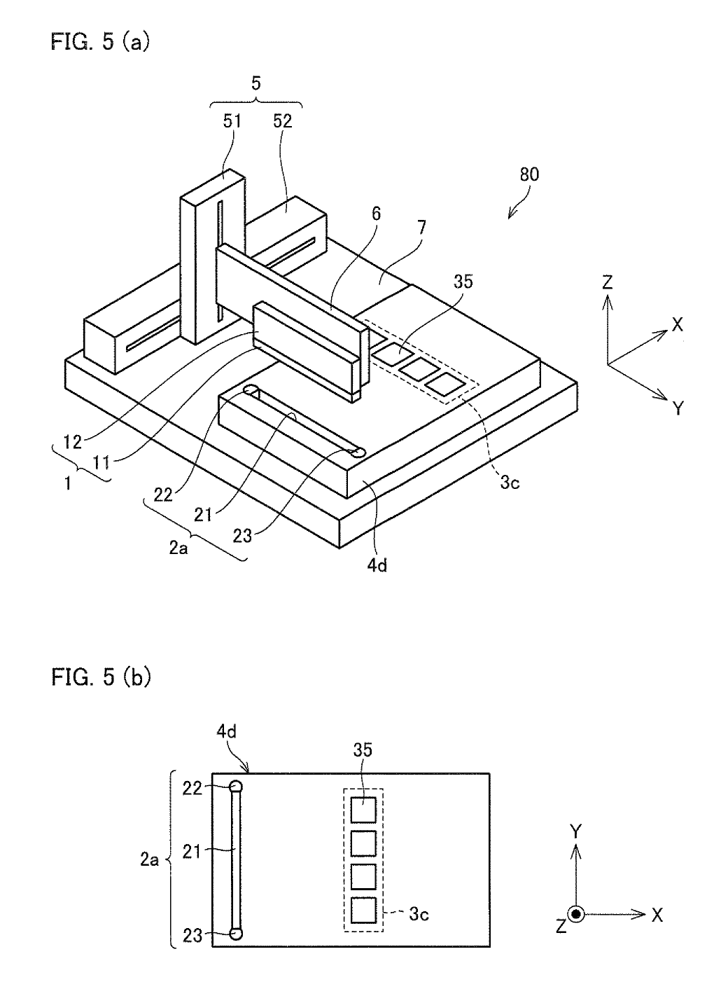

One embodiment of a sample analysis apparatus according to the present invention is described below, referring to FIG. 5. A sample analysis apparatus 80 according to the present embodiment is an apparatus for carrying out all the steps from first analysis (isoelectric focusing electrophoresis in a liquid) to second analysis by one apparatus continuously. Main configuration thereof is illustrated in FIG. 5(a). In the present embodiment, members identical with those in the first to third embodiments are labeled in the same manner.

The sample analysis apparatus 80 according to the present embodiment performs the isoelectric focusing electrophoresis in the liquid filled in a first analysis section 2a, collects sample components by a sample collecting section 11, and then extracts the collected sample components into a buffer solution held in an introducing section 3c of a second analysis section. That is, the first analysis of the present embodiment uses a liquid analysis medium.

As illustrated in FIG. 5(a), the sample analysis apparatus 80 according to the present embodiment includes a sample collecting tool 1 for collecting the sample components after the first analysis and providing the sample components to the second analysis. The sample collecting tool 1 includes a sample collecting section 11 and a holding section 12. In the present embodiment, the sample collecting section 11 is a dry gel, which is a liquid absorbing material, and has a function of collecting the sample components physically.

The sample analysis apparatus 80 according to the present embodiment further includes the first analysis section 2a and the introducing section 3c of the second analysis section. The first analysis section 2a includes: a first migration vessel 21, in which the first analysis medium is to be introduced, for performing the isoelectric focusing electrophoresis in the liquid; a reservoir 22 for keeping an anode solution; and a reservoir 23 for keeping a cathode solution. The first migration vessel 21 is thin enough to perform capillary electrophoresis. The introducing section 3c of the second analysis section also acts as an introducing section for introducing the sample for further analysis of the sample components after the first analysis. The introducing section 3c includes plural sample treating vessels 35. Even though it is not illustrated here, tubes are respectively connected to the sample treating vessels 35, the tube transporting the liquid sample from each sample treating vessel 35 to the second analysis section main body (not illustrated).

As illustrated in FIG. 5, the first analysis section 2a and the introducing section 3c of the second analysis section are provided on a substrate 4d disposed on a plate 7. On the plate 7, driving means 5 is disposed for driving the sample collecting tool 1. The driving means includes a vertical direction driving stage 51 and a horizontal driving stage 52. The sample collecting tool 1 is attached to a supporter 6 connect to the driving means 5, whereby the sample collecting tool 1 can be moved in the X and Z directions in FIG. 5(a).

With the sample analysis apparatus of the present embodiment having this arrangement, it is possible to continuously perform all the steps from the first analysis to the second analysis by only one apparatus as described below.

The first analysis is similar to that in the first embodiment. After the first analysis, the supporter 6 is moved in the X direction of FIG. 5(a) to a position right above the first analysis section 2a by the horizontal direction driving stage 52 of the driving means 5. Then, the supporter 6 is lowered in the Z direction of FIG. 5(a) by the vertical direction driving stage 51, thereby contacting the sample collecting section 11 of the sample collecting tool 1 with the first analysis medium in the first migration vessel 21. The sample collecting section 11 in contact with the first analysis medium in the first migration vessel 21 collects (absorbs) the analysis medium and sample components, which have been analyzed in the first analysis medium in the first migration vessel 21. Then, the supporter 6 moves in the X and/or Z direction of FIG. 5(a) by the vertical direction driving stage 51 and the horizontal driving stage 52, and then the sample collecting section 11 is inserted into the buffer solution held in the sample treating vessel 35 at the introducing section 3c of the second separating section.

The sample collecting tool 1 according to the present embodiment includes a sample collecting section 11 and a holding section 12 as illustrated in FIG. 6(c). The arrangement of the sample collecting section 11 makes it possible to provide fractions of the sample components independently to different (or same) second analysis after the first analysis.

The sample treating vessel 35 acts as a treating vessel in which the sample components analyzed by the first analysis are released (extracted) from the sample collecting section 11, and also acts as the sample introducing section for providing the sample components to the second analysis. The sample treating vessel 35 holds a buffer solution preferable for the adopted second analysis. The extraction of the sample in the sample treating vessel 35 may be facilitated by shaking the sample collecting section 11 inserted in the sample treating vessel 35 vigorously, heating the sample collecting section 11, utilizing a difference in osmosis pressure of the buffer solution, or the like. Moreover, in case where the sample components are electrostatic, voltage application across the sample treating vessel 35 facilitates the dissolution of the sample components from the sample collecting section 11 into the buffer solution. Regarding the electrodes for applying the voltage, a person skilled in the art who read this Specification will easily understand where to locate the electrodes, which kinds of the electrodes are applicable, etc.

The buffer solution containing the sample components extracted from the sample collecting section 11 is transported to a main body of the second analysis section via the tube connected to the sample treating vessel 35, and analyzed therein (second analysis).

As to the second analysis section of the present embodiment, any analysis (e.g., mass spectrometry, chromatography, and the like) conventionally known in this field are applicable, provided that the analysis analyzes a liquid sample.

Variations of the constituent members of the sample analysis apparatus 80 according to the present embodiment are described below.

<Substrate 4d and Sample Treating Vessel 35>

In case where the sample components bind with the sample collecting section 11 via the affinity binding, the buffer solution for the extraction has a composition that creates an environment (e.g., low pH environment) dissociates the sample components from the sample collecting section 11.

Between the first analysis section 2a and the introducing section 3c of the second analysis section, two or more sample treating vessels structurally similar to the sample extracting vessel 35 may be provided. The sample treating vessel may be provided with protease, bromocyane, or the like so as to peptize the sample components (proteins). Moreover, because the first analysis of the present embodiment is an isoelectric focusing electrophoresis, the sample may be treated with a surfactant having an electric charge, so as to electrify the sample components.

<Other Preferable Arrangements>

It is preferable that the sample analysis apparatus 80 is connected to a power source. This makes it possible to supply power to the first analysis section 2a, and the plural extracting section 35, thereby making it possible to carry out the sample analysis automatically. Moreover, the sample analysis apparatus 80 may be connected to a computer, in which a control program is installed for controlling the analysis procedure of the sample analysis apparatus 80. With this arrangement, the sample analysis apparatus 80 according to the present embodiment can perform the following actions automatically:

separating the sample components (proteins) according to the electric charges thereof by the isoelectric focusing electrophoresis performed in the first analysis section 2a;

collecting the separated sample components by the sample collecting section 11; and

extracting the collected sample components into the sample extracting vessels 35 with the result of the first analysis maintained.

Because all the procedures of the analysis in the sample analysis apparatus 80 can be performed under automatic control, the sample analysis can be carried out with higher safety and reproducibility.

With the sample analysis apparatus 80, desired fractions of the sample components after the first analysis can be provided to the second analysis efficiently without mixing them.

### 2-5: Fifth Embodiment of Sample Analysis Apparatus

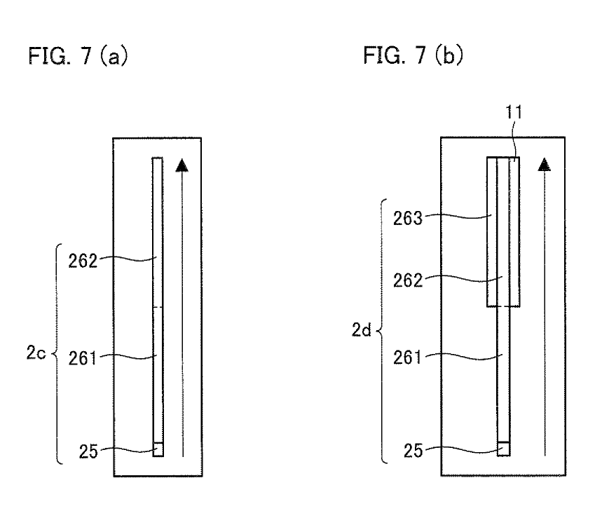

One embodiment of a sample analysis apparatus according to the present invention is described below, referring to FIG. 7. The sample analysis apparatus according to the present embodiment is an apparatus for carrying out all the steps from the first analysis to the second analysis by one apparatus continuously. A configuration of a first analysis section 2c and 2d is illustrated in FIGS. 7(a) and 7(b). In the present embodiment, merely the variation of the first analysis is discussed. A person skilled in the art, who reads this Specification, will easily understand the configuration of the second analysis which follows the first analysis.

The first analysis may use a liquid analysis medium or a solid analysis medium. In case of the solid analysis medium, the present embodiment is not limited to a gel discussed in the third embodiment. If the analysis medium is not electrically insulating (that is, it contains an electrolyte), the analysis medium can be applied to the present invention in combination with voltage application. In the present embodiment, the first analysis medium for the first analysis is a solid (stationary phase of the chromatography) by way of example.

<First Analysis Section 2c>

The first analysis section 2c according to the present embodiment is configured to perform liquid chromatography. As illustrated in FIG. 7(a), the first analysis section 2c includes a cartridge-filled vessel 261 filled with a cartridge (silica gel to whose surface a function group is bonded) and an elution vessel for receiving eluted sample components. A sample introducing section 25 for introducing the sample in the cartridge-filled vessel 261 is provided at one end of the cartridge-filled vessel 261, so that the sample introducing section 25 can supply the sample to be analyzed, into the cartridge-filled vessel 261. An analyte solution containing the sample components (for example, phosphate buffer, or the like) is flown in the direction of the arrow in FIG. 7 by a pump (not illustrated) linked to the sample introducing section 25 via a tube. Moreover, an end of the elution vessel 262 (the end thereof on the arrow-head side) is linked with a pump (not illustrated) via a tube. This pump sucks up the analyte solution flowing to the elusion vessel 262.

After the sample introduced in the sample introducing section 25 is analyzed in the cartridge-filled vessel 261, the sample components are eluted into the elution vessel 262 respectively. To collect the sample from the first analysis section 2c illustrated in FIG. 7(a), the sample collecting section 11 of the present embodiment is a dry gel (not illustrated), which is a liquid absorbing material, and has a function of collecting the sample components physically, in the present embodiment. Refer to the first embodiment etc. for the explanation on the collecting of the sample using a dry gel as the sample collecting section 11.

The first analysis section for performing the liquid chromatography can be changed as appropriate according to the configuration of the sample collecting section 11. For example, in case where the sample collecting section 11 is a PVDF film for adsorbing (collecting) the sample components by their electrostatic properties, the first analysis may have a configuration as illustrated in FIG. 7(b).

In FIG. 7(b), an electrode 263 is further provided on one side of the elusion vessel 262. On another side of the elution vessel 262, the sample collecting section made of a PVDF film is disposed. With this arrangement, voltage application of the elution vessel 262 causes the eluted sample components to transfer to the sample collecting section 11. Refer to the second embodiment etc. for the explanation on the collecting of the sample using a PVDF film as the sample collecting section 11.

The sample collecting section 11 in the present embodiment is not limited to the dry gel or PVDF film, and it is also preferable that the sample collecting section 11 is made of a metal such as gold, platinum, or the like, or a electrically conductive metal oxide such as ZrO₂. With such a sample collecting section, it is easy to collect proteins (sample components) by adsorbing the proteins via the thiol group thereof with the sample collecting section. Moreover, such a sample collecting section can act as an electrode, and is suitable for use in transferring the sample components by voltage application.

The present embodiment discusses the case where the sample is collected from the. However, it is also possible to collect the sample from a solid by using the sample collecting section 11 as an electrode.

Moreover, the present embodiment is arranged such that the cartridge, which is the analysis medium, is a silica gel to surface of which the functional group is bonded. The functional group may be changed according to the sample components to be analyzed. Moreover, depending on the type of the chromatography to carry out (e.g., gel filtration, ion exchange, affinity, hydrophobic interaction, reverse phase, demineralization/buffer exchange, chromatography focusing, etc.), the type of the cartridge may be selected. Examples of the cartridge other than the silica gel include C18 cartridge for use in the reverse phase chromatography, TBA chloride cartridge for use in ion exchange chromatography, and the like.

The pump to flow the analyte solution may be any pump known in this field (e.g., syringe pump, Peristaltic pump, Diaphragm pump). Moreover, in order to adjust the quantity of the analyte solution to be sent to the first analysis section, a valve may be provided at the tube connecting the pump and the sample introducing section 25.

### Example 1

A sample collecting tool and a sample analysis apparatus according to the present invention were prepared and used to perform gel 2-dimensional electrophoresis separation of proteins in which a sample was proteins and a first analysis was liquid isoelectric focusing electrophoresis.

A sample collecting section 11 of the sample collecting tool 1 was a pH gradient solidified gel (dried) on a film. The gel was made by GE healthcare Bio-Sciences K.K., and cut in 0.5 mm in width and 50 mm in length. Moreover, a holding section 12 as PMMA, processed to 0.5 mm in width, 50 mm in length, 15 mm in thickness. A bottom surface of the holding section 12 and a film surface of the pH gradient solidified gel were attached with each other, thereby to prepare the sample collecting tool 1.

Moreover, a 2-dimensional electrophoresis substrate 4a as illustrated in FIG. 1 was prepared from PMMA. A first migration vessel 21 was 0.5 mm in width, 50 mm in length, and 1 mm in thickness. On each end of the first migration vessel 21, a reservoir 22 for anode solution and a reservoir 23 for cathode solution were prepared respectively, which were 2 mm in diameter and 1 mm in thickness. A surface of the first migration vessel 21 was treated with phospholipids. The sample treating vessel 41 was prepared, which was 1 mm in width, 50 mm in length, and 1 mm in thickness. Moreover, buffer solution vessels 32 and 33 were prepared, which were 10 mm in width, 50 mm in length, and 5 mm in thickness, and which were used as reservoirs for anode solution and cathode solution for SDS-PAGE, respectively. Between the reservoirs for SDS-PAGE, a second migration vessel 31 was prepared, which was 50 mm in width, 50 mm in length, and 1 mm in thickness. Another substrate was provided as a lid to seal the second migration vessel 31.

A driving means 5 was assembled on a plate 7 from a commercially available vertical direction driving stage 51 and a horizontal direction driving stage 52. Then, a supporter made of PMMA was connected to the vertical direction driving stage 5. To the supporter 6, the sample collecting tool 1 was adhered. After that, the 2-dimensional electrophoresis substrate 4a was attached to the plate 7. Thereby, the sample analysis apparatus 10 was prepared. Into the reservoirs 22 and 23 on each end of the first migration vessel 21 of the 2-dimensional electrophoresis substrate 4a, and the buffer solution vessel 32 and 33 for SDS-PAGE, electrodes were inserted respectively, each of which was connected to power source for the electrophoresis. The driving means 5 and the power source were connected to a computer.

A phosphate solution was introduced into the reservoir 22, while a sodium hydroxide solution was introduced into the reservoir 23. Then, an analyte solution a protein sample and ampholytic carrier (Ampholine of GE healthcare Bio-Sciences K.K.) buffer solution were introduced in the first migration vessel 21. Into the sample treating vessel 41, equilibrium solution made of SDS, Tris-HCL, and DTT was introduced. Into the buffer solution vessels 32 and 33 on both the ends, a migration buffer solution made of SDS, Tris, and Glysine was introduced.

After these solutions were introduced, the sample analysis apparatus 10 was automatically driven by a control program implemented in the computer. The sample analysis apparatus 10 was controlled in the following manner.

Firstly, the vertical direction driving stage 51 was driven to insert the sample collecting section 11 into the first migration vessel 21. Then, the first migration vessel 21 was sealed with the lid. A voltage (2500V) was applied across the electrodes inserted in the reservoirs 22 and 23 of the first migration vessel 21, thereby to carry out the liquid isoelectric focusing electrophoresis. After 5 min, the vertical direction driving stage 51 was driven while the voltage application was carried on, so as to insert the sample collecting section 1 into the analysis solution. Further, while the voltage application was carried on, separation was carried out for 5 min. Then, the vertical direction driving stage 51 was driven to hoist the sample collecting section 1. Then, the voltage application was stopped. It was visually confirmed that the analyte solution in the first migration vessel 21 was collected in the sample collecting section 1 (the dry gel absorbed the analyte solution and swelled) (first analysis and sample collecting).

Next, the horizontal direction driving stage 52 was driven to transport the sample collecting section 1 to a position above the sample treating vessel 41 in which the equilibrium solution was held. After the sample collecting section containing the sample components was immersed in the equilibrium solution by driving the vertical direction driving stage 51, the vertical directing driving stage 51 was driven up and down (shaking action) thereby to attain equilibrium of the sample (5 min).

After the equilibrium operation, the vertical direction driving stage 51 and the horizontal direction driving stage 52 were driven to transport the sample collecting section 1 containing the sample components to an end surface of the gel for SDS-PAGE. After the transportation, a voltage was applied across the electrodes inserted in the buffer solution vessels 32 and 33 for SDS-PAGE. Thereby, molecular weight separation of the proteins was carried out by SDS-PAGE (30 mA constant current, 20 min). After the migration was finished, the voltage was stopped. Then, the sample collecting section 1 was hoisted by driving the vertical direction driving stage 51. Then, the gel 2-dimensional electrophoresis migration was finished.

After the end of the analysis, the gel for SDS-PAGE was taken out and stained with CBB. This showed good separation of the proteins according to their isoelectric points and molecular weight. That is, the use of the present invention attains high-speed and automatic gel 2-dimensional electrophoresis of proteins.

### Example 2

A sample collecting tool 1 and sample analysis apparatus 70 according to the present invention were prepared, with which soluble fractions of mouse liver containing HisG positope control protein was analyzed by SDS-PAGE as first analysis and separation (western blotting) using affinity binding reaction.

The sample collecting section 11 of the sample collecting tool 1 was a commercially available PVDF film cut into a size of 0.5 mm in width, 50 mm in length. Moreover, a holding section 12 was PMMA processed to 0.5 mm in width, 50 mm in length, and 15 mm in thickness. A side of the holding section 12 and one side of the PVDF film were adhered to make the sample collecting tool 1.

Further, a first analysis medium 8 of an analysis medium transport tool 1′ was a separation gel (acrylamide gel) formed in a size of 0.5 mm in width and 50 mm in length. A holding section 12′ was PMMA and processed in a size of 0.5 mm in width, 50 mm in length, and 15 mm in thickness. A bottom surface of the holding section 12′ and one side of the separation gel were adhered thereby to make the analysis medium transport tool 1′.

From PMMA, a substrate 4a as illustrated in FIG. 3(b) was prepared. A migration vessel 24 was 0.5 mm in width, 50 mm in length, and 1 mm in thickness. On each end of the migration vessel 24, a reservoir 22 for an anode solution and a reservoir 23 for a cathode solution were prepared, which was 2 mm in diameter and 1 mm in thickness. A surface of the migration vessel 24 was treated with phospholipids. At that end portion of the migration vessel 24 at which the reservoir 23 for the anode solution was located, a sample introducing section 25 was formed. Six sample treating vessels 3b (34a to 34f) were provided, which were 1 mm in width, 50 mm in length, and 1 mm in thickness. Between the migration vessel 24 on the substrate 4c and the sample treating vessel 3b, a sample transfer vessel 42 (5 mm in width, 5 mm in length, 1 mm in thickness) was prepared, in which a buffer solution for sample transfer was contained. In the sample transfer vessel 42, electrodes for applying a voltage necessary for transfer of sample components were provided. On another portion of the substrate 4c, a transfer film storage section 43 (1 mm in width, 50 mm in length, 1 mm in thickness) was provided for storing the sample collecting tool 1 before the analysis. In the sample transfer vessel 42 and the transfer film storage section 43, a transfer buffer solution (made of Tris, Glysine, and Methanol) was contained.

A driving means 5 was assembled on a plate 7 from a commercially available vertical direction driving stage 51 and a horizontal direction driving stage 52. Then, a supporter made of PMMA was connected to the vertical direction driving stage 5. To the supporter 6, the sample collecting tool 1 was adhered. After that, the substrate 4c was attached to the plate 7. Thereby, the sample analysis apparatus 70 was prepared. Into the reservoirs 22 and 23 on each end of the first migration vessel 21 of the substrate 4c, and the buffer solution vessel 32 and 33 for SDS-PAGE, electrodes were inserted respectively, each of which was connected to power source for the electrophoresis. The driving means 5 and the power source were connected to a computer.

After an electrolyte solution (a solution made of Tris, Glysine, SDS) for electrophoresis was contained in the reservoirs 22 and 23 on the respective ends of the migration vessel 24, a blocking solution (1% BSA/PBST (Phosphate Buffer Saline Tween-20)) was introduced in the sample treating vessel 34a, and a washing solution (PBST) was introduced in the sample treating vessel 34b. In the sample treating vessel 34c, a primary antibody solution prepared with PBST was introduced. Into the sample treating vessel 34d, the washing solution (PBST) was introduced. Into the sample treating vessel 34e, a fluorescence labeled secondary antibody solution prepared with PBST was introduced. Into the sample treating vessel 34f, the washing solution (PBST) was introduced.

After these solutions were introduced, the sample collecting tool 1 was stored in the transfer film storage section 43 in advance, so that the sample collecting tool 1, adhered to the supporter 6 by vacuum suction, was to be transported by driving the driving tool 5 after the first analysis. Then, the sample analysis apparatus 70 was automatically driven by a control program implemented on the computer, and controlled in the following manner.

To begin with, the analysis medium transport tool 17 was sucked to the supporter 6 by vacuum suction. By driving the vertical direction driving stage 51 and horizontal direction driving stage 52, the first analysis medium 8 was transported to a position above the migration vessel 24.

By driving the vertical direction driving stage 51, the analysis medium transport tool 1′ was lowered so as to put the first analysis medium 8 in the migration vessel 24. After proteins sample treated with SDS was introduced in the sample introducing section 25, a voltage (200V for 30 min) was applied across the electrodes inserted in the reservoirs 22 and 23 on the respective ends of the migration vessel 24. By this, the sample components (proteins) were separated in the first analysis medium 8 according to their molecular weights (SDS-PAGE). After SDS-PAGE was finished, the voltage application was stopped (first analysis).

The analysis medium transport tool 1′ was hoisted by driving the vertical direction driving stage 51, then moved by the vertical direction driving stage 51 and the horizontal driving stage 52 so as to transport the first analysis medium 8 into the sample transfer vessel 42. After the first analysis medium 8 was placed on a fixing section formed in the sample transfer vessel 42, the vacuum suction was released thereby to separate the analysis medium transport tool 1′ and the supporter 6.

By driving the vertical direction driving stage 51 and the horizontal direction driving stage 52, the supporter 6 was transported to a position above the transfer film storage section 43, at which the same collecting tool 1 and the supporter 6 could contact with each other. After adhering the sample collecting tool 1 to the supporter 6 by vacuum suction, the sample collecting tool 1 was transported by driving the vertical direction driving stage 51 and the horizontal direction driving stage 52, thereby to insert the sample collecting section 11 into the sample transfer vessel 42. While driving the driving means 5, the first analysis medium 8 of the analysis medium transport tool 1′ and the sample collecting section 11 of the sample collecting tool 1 were adhered. By applying a voltage across the electrodes provided to the sample transfer vessel 42, the transfer of the sample components was carried out.

By driving the vertical direction driving stage 51 and the horizontal direction driving stage 52, the sample collecting tool 1 having the sample collecting section 11 to which the sample components were transferred was transported to a position above the sample treating vessel 34a. By driving the vertical direction driving stage 51, the sample collecting section 11 was immersed in the blocking solution. Then, the sample collecting tool 1 was moved up and down (shacking action) by the vertical direction driving stage 51, so as to perform the blocking treatment (30 min). After the blocking treatment, the sample collecting section 11 was transported to the sample treating vessel 34b by driving the vertical direction driving stage 51 and the horizontal direction driving stage 52, so as to immerse and shake the sample collecting section 11 therein in the similar manner (10 min). Similar operations were carried out in the sample treating vessels 34c to 34f. Thereby, the reaction of the proteins in the sample collecting section 11 with the primary antibody (anti-HisG antibody (mouse monoclonal antibody) was carried out for 1 hours followed by 10 min washing, and the reaction of the primary antibody with the fluorescence labeled secondary antibody (goat anti-mouse antibody labeled with Alexa Fluor 488) was carried out for 30 min followed by 10 min washing. After all the operations were finished, the sample collecting tool 1 was hoisted by driving the vertical direction driving stage 51, whereby the western blotting was finished.

After the western blotting, the sample was detected with an optical system provided with a light source (filtered lamp) and a CCD camera. The detection confirmed the existence of the targeted protein. That is, the use of the sample analysis apparatus of the present invention carried out the separation of the protein by SDS-PAGE and the detection by western blotting, automatically.

The present invention is not limited to the description of the embodiments above, but may be altered by a skilled person within the scope of the claims. An embodiment based on a proper combination of technical means disclosed in different embodiments is encompassed in the technical scope of the present invention.

All the patent documents cited in this Specification are incorporated herein by reference

The sample analysis method according to the present invention may further include removing the collected sample component from the sample collecting section.

With this arrangement, the second analysis may be such analysis that cannot analyze the sample components kept in the sample collecting section.

The sample analysis according to the present invention preferably further includes providing electrodes respectively to the first analysis medium and the sample collecting section.

With the arrangement in which the sample analysis according to the present invention preferably further includes this step, it is possible to apply a voltage across the first analysis medium and the sample collecting section in the step of collecting the sample. With this, the sample components having electric charges and having been analyzed by the first analysis can be collected efficiently.

The sample analysis method according to the present invention is preferably arranged such that the first analysis medium is a liquid.

With this arrangement, it is possible to easily collect the sample components into the sample collecting section from the first analysis medium after the first analysis.

The sample analysis method according to the present invention may further include evaporating the liquid while performing the step of collecting.

By evaporating the first analysis medium, the sample is concentrated after the first analysis. This makes it possible to collect the sample more efficiently.

The sample analysis method according to the present invention preferably further includes pre-treating the sample, which is to be analyzed by the first analysis. The step of pre-treating may be a step of treating the sample with a reagent that makes it easy to collect the sample from the first analysis medium, or may be a step of treating the sample with a reagent that puts the sample in a condition suitable for performing the first analysis.

With this arrangement, the collecting of the sample can be efficiently carried out, and various analysis techniques can be adopted as the first analysis.

Further, the sample analysis method according to the present invention preferably further includes pre-treating the collected sample components, which are to be provided to the second analysis. The step of pre-treating may be a step of treating the sample with a reagent that puts the sample in a condition suitable for performing the first analysis.

With this arrangement, various analysis techniques can be adopted as the second analysis.

Moreover, the sample analysis method according to the present invention is preferably arranged such that the sample collecting section includes a metal.

With this arrangement, the sample collecting section can be used as an electrode, thereby making it possible to effectively collect the sample having electric charges.

Moreover, the sample analysis method according to the present invention may be arranged such that the sample collecting section includes (i.e., is made of or is provided with) a liquid absorbing material.

With this arrangement, it is possible to collect the sample components from a liquid to the sample collecting section.

Moreover, the sample analysis method according to the present invention may be arranged such that the sample collecting section includes an electrostatic material or is electrostatic.

With this arrangement, the sample components having an electrostatic property (having electric charges, etc.) can be bound to or adsorbed to the sample collecting section.

Further, the sample analysis method according to the present invention may be arranged such that the sample collecting section includes a hydrophilic or hydrophobic material, or is hydrophilic or hydrophobic.

With this arrangement, hydrophilic or hydrophobic sample components can be bound to or adsorbed to the sample collecting section.

Moreover, the sample analysis method according to the present invention may be arranged such that the sample collecting section includes (i.e., is made of or is provided with a material affinitive with the sample components to be collected.

With this arrangement, the sample components affinitive with the sample collecting section can be bound to or adsorbed to the sample collecting section.

Furthermore, the sample analysis method according to the present invention may be arranged such that the sample collecting section has a functional group.

With this arrangement, the sample components reactive or affinitive with the functional group can be bound to or adsorbed to the sample collecting section.

Moreover, a sample analysis apparatus according to the present invention is an apparatus for analyzing a sample in plural times continuously, the sample containing plural sample components, the apparatus including: a first analysis section in which a first analysis medium is held; a sample collecting section for collecting sample components analyzed by the first analysis; and a second analysis section for analyzing the sample components collected by the sample collecting section.

With this arrangement, the sample analysis analyzed by the first analysis can be collected in the sample collecting section with the result of the analysis maintained. Thus, simply by moving the sample collecting section, the sample components analyzed by the first analysis can be provided to the second analysis with the result of analysis maintained. To provide to the second analysis may be inserting or contacting the sample collecting section in the second analysis section, or removing the collected sample and supplying it to the second analysis section.

By arranging such that the means are operated automatically under control of a computer to which they are connected, it is possible to analyze one sample plural times continuously with a little need of manual operation. Thus, it is possible to obtain analysis result with high reproducibility even among different users.

The apparatus according to the present invention attains similar effects as the sample analysis method according to the present invention.

The sample analysis apparatus according to the present is preferably arranged such that the first analysis medium and the sample collecting section are connected to voltage applying means.

With this arrangement, it is possible to apply a voltage across the first analysis medium and the sample collecting section, thereby making it possible to efficiently collect the sample, which has been analyzed by the first analysis and has electric charges.

Moreover, the sample analysis apparatus according to the preset invention may further include: temperature adjusting means for adjusting a temperature of the first analysis medium.

For example, if the temperature adjusting means is for increasing the temperature of the first analysis medium, this means may be used to evaporate the liquid analysis medium. Thus, this makes it possible to efficiently collect the sample analyzed by the first analysis.

The sample analysis apparatus according to the present invention preferably further includes driving means for moving the sample collecting section.

This arrangement allows more operations of the sample analysis to be performed automatically, and consequently reduces tedious manual operations.

Moreover, the sample analysis apparatus according to the present invention is preferably arranged such that the sample collecting section includes a metal.

With this arrangement, the sample collecting section can be used as an electrode, thereby making it possible to effectively collect the sample having electric charges.

Furthermore, the sample analysis apparatus according to the present invention may further include a first sample pre-treating vessel for pre-treating the sample, which is to be analyzed by the first analysis section. The first sample pre-treating vessel may be a vessel for treating the sample with a reagent that makes it easier to collect the sample from the first analysis medium, or a vessel for treating the sample with a reagent that puts the sample in a condition suitable for the first analysis.

With this arrangement, the collecting of the sample can be efficiently carried out, and various analysis techniques can be adopted as the first analysis.

Further, the sample analysis apparatus according to the present invention may further include a second sample pre-treating vessel for pre-treating the collected sample components, which are to be provided to the second analysis section. The second sample pre-treating vessel may be a vessel for treating the sample with a reagent that puts the sample components in a condition suitable for the second analysis. Moreover, in the second sample pre-treating vessel, the sample component analyzed by the first analysis may be collected by using the collecting section.

With this arrangement, various analysis techniques can be adopted as the second analysis.

Moreover, the sample analysis apparatus according to the present invention may be arranged such that the sample collecting section includes (i.e., is made of or is provided with) a liquid absorbing material.

With this arrangement, it is possible to collect the sample components from a liquid to the sample collecting section.

Furthermore, the sample analysis apparatus according to the present invention may be arranged such that the sample collecting section includes (i.e., is made of or is provided with) an electrostatic material.

With this arrangement, the sample components having an electrostatic property (having electric charges, etc.) can be bound to or adsorbed to the sample collecting section.

Moreover, the sample analysis apparatus according to the present invention may be arranged such that the sample collecting section includes a hydrophilic or hydrophobic material, or is hydrophilic or hydrophobic.

With this arrangement, hydrophilic or hydrophobic sample components can be bound to or adsorbed to the sample collecting section.

Furthermore, the sample analysis apparatus according to the present invention may be arranged such that the sample collecting section includes (i.e., is made of or is provided with) a material affinitive with the sample components to be collected.

With this arrangement, the sample components affinitive with the sample collecting section can be bound to or adsorbed to the sample collecting section.

Moreover, the sample analysis apparatus according to the present invention may be arranged such that the sample collecting section has a functional group.

With this arrangement, the sample components reactive or affinitive with the functional group can be bound to or adsorbed to the sample collecting section.

Moreover, the sample analysis method according to the present invention includes performing first analysis of a sample in a liquid; collecting the analyzed sample by a solid sample collecting section; and performing at least one second analysis with the sample collected in the solid sample collecting section.

In this arrangement, the first analysis is carried out in a liquid at a high rate, and the analyzed sample is collected by the solid sample collecting section. With this arrangement, it is possible to provide the sample to the second analysis via the sample collecting section.

Moreover, the sample analysis method according to the present invention is preferably arranged such that the first analysis performed in the liquid is an electrophoresis.

The electrophoresis performed in the liquid is preferably isoelectric focusing electrophoresis.

The use of the isoelectric focusing electrophoresis concentrate the sample in narrow bands and makes it possible to analyze with high separating ability.

Moreover, the sample analysis method according to the present invention is preferably arranged such that at least one of the second analysis is one of electrophoresis, affinity binding reaction, mass spectrometry, and chromatography.

Moreover, the sample analysis method according to the present invention is preferably arranged such that the electrophoresis adopted as the second analysis is molecular weight fractionation electrophoresis.

With this arrangement, it is possible to attain analysis (2-dimensional electrophoresis) based on the isoelectric points and molecular weight with high separating ability.

Moreover, the sample analysis method according to the present invention is preferably arranged such that the affinity binding reaction adopted as the second analysis is an immune reaction.

In case where proteins are analyzed with this arrangement, it is possible to detect specific proteins by immune reaction after the first analysis.

Moreover, the sample analysis method according to the present invention is preferably arranged such that the sample collecting section for collecting the sample analyzed by the first analysis is made of a liquid absorbing material, a polymer film, or a substrate material.

The sample analysis method according to the present invention is preferably arranged such that the liquid absorbing material of the sample collecting section is a material selected from the group consisting of a dry gel, gel, pulp material, and filter paper, which is swellable/absorbable with an aqueous or non-aqueous liquid.

With this arrangement, it is possible to collect the analyzed sample in the liquid by sample collecting section instantly, and to start the second analysis such as molecular weight fractionation electrophoresis smoothly.

Moreover, the sample analysis method according to the present invention is preferably arranged such that the polymer film of the sample collecting section is a made of a material selected from the group consisting of PVDF, nitrocellulose, nylon, Teflon, Zitex, polypropylene, polytetrafluoroethylene, cellulose acetate, and latex.

With this arrangement, it is possible to perform western blotting and the detection of specific protein by using the affinity binding reaction such as immune reaction, by using the sample collecting section in which the sample is collected.

The sample analysis method according to the present invention is preferably arranged such that a substrate material of the sample collecting section is a material selected from the group consisting of PMMA, polyethylene, polystyrene, PET, COP, polycarbonate, vinylchloride, glass, stainless, DLC, and ceramic.

With this arrangement, it is possible to perform mass spectrometry analysis by using the sample collecting section in which the sample is collected. Moreover, for example, it is possible to prepare a protein chip with proteins analyzed by the first analysis.

Moreover, the sample analysis method according to the present invention is preferably arranged such that the sample collecting section is hydrophobic or converted to be hydrophobic.

This arrangement contributes to the efficiency in collecting, on the sample collecting section, the sample (e.g., protein) analyzed by the first analysis.

Furthermore, the sample analysis method according to the present invention is preferably arranged such that the sample collecting section has a functional group for fixing the sample on the sample collecting section.

With this arrangement, it is possible to collect, for example, the proteins by reacting the proteins with the functional group on the sample collecting section, to use the sample collecting section as a protein chip.

Moreover, the sample analysis method according to the present invention is preferably arranged such that an affinity binding material (for example, antibody) is immobilized on the sample collecting section.

With this arrangement, it is possible to collect on the sample collecting section only the specific protein analyzed by the first analysis, and to detect the specific proteins by immune reaction after the first analysis.

Moreover, the sample analysis method according to the present invention is preferably arranged such that the sample collecting section is attached to a holding substrate.

With this arrangement, it is possible to use a thin or fragile sample collecting section stably.

Moreover, the sample analysis method according to the present invention may be arranged such that the sample collecting section may be attached to part of a holding substrate.

With the arrangement, part of the sample analyzed by the first analysis is collected by the sample collecting section and used for further analysis. Thus, the first analysis can be used for fractioning of the sample (pretreatment).

Moreover, the sample analysis method according to the present invention includes: performing first analysis of a sample in a liquid; collecting the analyzed sample in the liquid by inserting the sample collecting section, which may be attached to the holding substrate; and removing the sample collecting section, which contains the collected sample.

The step of collecting the sample preferably includes evaporating the liquid.

With this arrangement, the sample analyzed by the first analysis can be effectively collected by the sample collecting section, regardless of the material of the sample collecting section.

Moreover, the sample analysis method according to the present invention includes performing isoelectric focusing electrophoresis of a sample as the first analysis in a liquid by applying a voltage in the liquid; while applying the voltage in the liquid, collecting the analyzed sample in the liquid by inserting the sample collecting section, which may be attached to the holding substrate; and removing the sample collecting section, which contains the collected sample.

With this arrangement, the first analysis is the isoelectric focusing electrophoresis using a liquid, which has a high speed and a high separating ability. While the voltage is applied in the liquid, the sample collecting section is inserted to collect the analyzed sample. This does not reduce the separating ability according to the isoelectric points at the collecting operation.

The sample analysis method according to the present invention may be arranged such that the step of collecting includes evaporating the liquid, in case the first analysis is the isoelectric focusing electrophoresis.

A sample collecting tool according to the present invention is a tool for collecting a sample from a liquid after first analysis of the sample is performed in the liquid, including a sample collecting section made of a liquid absorbing material, a polymer film or a substrate material.

Further, the sample collecting tool according to the present invention is preferably arranged such that the liquid absorbing material of the sample collecting section is a material selected from the group consisting of a dry gel, gel, pulp material, and filter paper, which is swellable/absorbable with an aqueous or non-aqueous liquid.

Moreover, the sample collecting tool according to the present invention is preferably arranged such that the polymer film of the sample collecting section is a made of a material selected from the group consisting of PVDF, nitrocellulose, nylon, Teflon, Zitex, polypropylene, polytetrafluoroethylene, cellulose acetate, and latex.

Moreover, the sample collecting tool according to the present invention is preferably arranged such that a substrate material of the sample collecting section is a material selected from the group consisting of PMMA, polyethylene, polystyrene, PET, COP, polycarbonate, vinylchloride, glass, stainless, DLC, and ceramic.

Moreover, the sample collecting tool according to the present invention is preferably arranged such that the sample collecting section is hydrophobic or converted to be hydrophobic.

Moreover, the sample collecting tool according to the present invention is preferably arranged such that the sample collecting section has a functional group for fixing the sample on the sample collecting section.

Moreover, the sample collecting tool according to the present invention is preferably arranged such that an affinity binding material (for example, antibody) is immobilized on the sample collecting section.

Moreover, the sample collecting tool according to the present invention is preferably arranged such that the sample collecting section is attached to a holding substrate.

Moreover, the sample collecting tool according to the present invention is preferably arranged such that the sample collecting section may be attached to part of a holding substrate.

A sample analysis apparatus according to the present invention includes: the sample collecting tool; a substrate provided with a first analysis section thereon for performing first analysis in a liquid which is contained in the first analysis section; a holding section for holding the sample collecting tool; and driving means for driving the holding section.

With this arrangement, the sample can be collected automatically after the first analysis, thereby making it possible to carry out the analysis more efficiently with better reproducibility.

The sample analysis apparatus according to the present invention preferably includes temperature adjusting means for adjusting a temperature of the liquid inside the first analysis section.

With this arrangement, it is possible to perform stable analysis by adjusting the temperature during the first analysis, and to evaporate the liquid by the heating at collecting the sample. This contributes to the efficiency in collecting the sample regardless of the material of the sample collecting section.

Moreover, the sample analysis apparatus according to the present invention preferably include voltage applying means for performing electrophoresis in the first analysis section.

Moreover, the sample analysis apparatus according to the present invention is preferably arranged such that the first analysis section has electrodes or a system for accepting insertion of electrodes.

Moreover, the sample analysis apparatus according to the present invention is preferably arranged such that the first analysis section is made hydrophilic.

With this arrangement, for example, a sample such as proteins will not be adsorbed to the first analysis section and can be collected by the sample collecting tool efficiently.

Moreover, the sample analysis apparatus according to the present invention is preferably arranged such that the first analysis is in a range of 1 to 5000 μm in width and depth.

With this arrangement, the analysis requires only a minute amount of sample, and heat generated by the electrophoresis can be radiated efficiently. Further, the evaporation of the liquid can be efficiently done by heating at the collecting the sample.

Moreover, the sample analysis apparatus according to the present invention is preferably arranged such that a level of the liquid is in a range of 1 to 1000 μm from a bottom of the first analysis section.

With this arrangement, the collecting the sample can be done instantly, and the evaporation of the liquid and collecting of the sample can be done efficiently.

Moreover, the sample analysis apparatus according to the present invention preferably includes: a first analysis section for performing the first analysis; a vessel for performing the second analysis; and at least one vessel for performing a treatment necessary for performing the second analysis.

With this arrangement, the second analysis can be done automatically and speedily after the first analysis. Further, this arrangement leads to miniaturization of the apparatus.

As described above, the use of the present invention makes it possible to use the analyzed sample components to another analysis with the result of the analysis maintained. Thus, it is possible to shorten the time necessary for performing plural times (kinds) of sample analysis. Further, it is possible to improve the analysis to give more accurate results. That is, the use of the present invention makes it possible to analyze one sample plural times efficiently.

Moreover, with the use of the present invention, the sample analyzed in a liquid by the first analysis can be used for the second analysis easily. Especially, it is possible to speed up the 2-dimensional electrophoresis in which a first stage analysis is isoelectric focusing electrophoresis. Moreover, it becomes easy to combine the capillary electrophoresis with another analysis method.

The embodiments and concrete examples of implementation discussed in the foregoing detailed explanation serve solely to illustrate the technical details of the present invention, which should not be narrowly interpreted within the limits of such embodiments and concrete examples, but rather may be applied in many variations within the spirit of the present invention, provided such variations do not exceed the scope of the patent claims set forth below.

What is claimed is:

1. A method of analyzing a sample plural times continuously, the sample containing plural sample components, the method comprising:
performing first analysis with the sample in a first analysis medium;
collecting the sample components after the first analysis with an analysis result of the first analysis maintained; and
providing the collected sample components to second analysis.

2. The method as set forth in claim 1, further comprising:
removing the collected sample component from the sample collecting section.

3. The method as set forth in claim 1, further comprising:
providing electrodes respectively to the first analysis medium and the sample collecting section.

4. The method as set forth in claim 1, wherein:
the first analysis medium is a liquid.

5. The method as set forth in claim 4, further comprising:
evaporating the liquid while performing the step of collecting.

6. The method as set forth in claim 1, further comprising:
pre-treating the sample, which is to be analyzed by the first analysis.

7. The method as set forth in claim 1, further comprising:
pre-treating the collected sample components, which are to be provided to the second analysis.

8. The method as set forth in claim 1, wherein:
the sample collecting section includes a metal.

9. The method as set forth in claim 1, wherein:
the sample collecting section includes a liquid absorbing material.

10. The method as set forth in claim 1, wherein:
the sample collecting section includes an electrostatic material.

11. The method as set forth in claim 1, wherein:
the sample collecting section includes a hydrophilic or hydrophobic material.

12. The method as set forth in claim 1, wherein:
the sample collecting section includes a material affinitive with the sample components to be collected.

13. The method as set forth in claim 1, wherein:
the sample collecting section has a functional group.

14. An apparatus for analyzing a sample in plural times continuously, the sample containing plural sample components, the apparatus comprising:
a first analysis section in which a first analysis medium is held;
a sample collecting section for collecting sample components analyzed by the first analysis; and
a second analysis section for analyzing the sample components collected by the sample collecting section.

15. The apparatus as set forth in claim 14, wherein:
the first analysis medium and the sample collecting section are connected to voltage applying means.

16. The apparatus as set forth in claim 15, further comprising:
temperature adjusting means for adjusting a temperature of the first analysis medium.

17. The apparatus as set forth in claim 14, further comprising:
driving means for moving the sample collecting section.

18. The apparatus as set forth in claim 14, further comprising:
a first sample pre-treating vessel for pre-treating the sample, which is to be analyzed by the first analysis section.

19. The apparatus as set forth in claim 14, further comprising:
a second sample pre-treating vessel for pre-treating the collected sample components, which are to be provided to the second analysis section.

20. The apparatus as set forth in claim 14, wherein:
the sample collecting section includes a liquid absorbing material.

21. The apparatus as set forth in claim 14, wherein:
the sample collecting section includes an electrostatic material.

22. The apparatus as set forth in claim 14, wherein:
the sample collecting section includes a hydrophilic or hydrophobic material.

23. The apparatus as set forth in claim 14, wherein:
the sample collecting section includes a material affinitive with the sample components to be collected.

24. The apparatus as set forth in claim 14, wherein:
the sample collecting section includes a metal.

25. The apparatus as set forth in claim 14, wherein:
the sample collecting section has a functional group.
